(19) United States
(12) Patent Application Publication
(10) Pub. No.: US 2025/0259567 A1
(43) Pub. Date: Aug. 14, 2025

(54) ANATOMICAL MODELS AND ASSOCIATED METHODS

(71) Applicant: ARTHREX, INC., Naples, FL (US)

(72) Inventors: Nick Metcalfe, Bonita Springs, FL (US); David Knopf, Bonita Springs, FL (US); Benjamin David Bernarding, Allison Park, PA (US); David Yang, Bedord, MA (US); Shervin F. Kazemi, Naples, FL (US); Gage Weimer, Ruskin, FL (US); Victoria Triaga, Naples, FL (US)

(21) Appl. No.: 19/048,337

(22) Filed: Feb. 7, 2025

Related U.S. Application Data
(60) Provisional application No. 63/551,565, filed on Feb. 9, 2024.

(51) Int. Cl. G09B 23/34; A61F 2/46; G09B 23/32

(52) U.S. Cl. CPC G09B 23/34; G09B 23/32; A61F 2/4684

(57) ABSTRACT

This disclosure relates to surgical systems, devices and methods for planning and implementing surgical procedures. The systems and methods disclosed herein may be utilized to establish physical anatomical models of anatomy. The physical anatomical model may include one or more features for viewing an interior of the model.

### CROSS-REFERENCED TO RELATED APPLICATION

This application claims priority to U.S. Provisional Application No. 63/551,565, which was filed on Feb. 9, 2024.

### BACKGROUND

This disclosure relates to surgical systems, devices and methods for planning and implementing surgical procedures utilizing physical models of anatomy.

Deformities may form along various bones and joints of the human musculoskeletal system. Patients may experience a fracture of one or more bones due to trauma. A surgeon may reduce the fracture and may secure the fragments of the bone with an implant to restore functionality to the patient. Surgeons may prepare for an orthopaedic surgery by performing a procedure on a cadaveric or saw bone specimen.

### SUMMARY

The systems and methods disclosed herein may be utilized to establish physical anatomical models of anatomy. The physical anatomical model may include one or more features for viewing an interior of the model.

A system for an orthopaedic procedure according to an implementation may include a physical anatomical model including a main body representative of anatomy. The main body may include a bone component. The bone component may include an exterior having a surface contour representative of a bone. The bone component may include a first portion establishing the exterior and a first material. The bone component may include a second portion adjacent to the first portion. The second portion may include a second material that may differ from the first material. The second material may transparent or translucent for viewing an interior of the bone component.

A system for a surgical procedure according to an implementation may include a physical anatomical model including a body associated with an anatomical profile of a bone. At least a first portion of the body may be formed of a first material that may be transparent or translucent, through which an interior of the body may be viewed. A light source may be operable to illuminate the interior of the body.

A method of rehearsing for a surgical procedure according to an implementation may include accessing a virtual anatomical model associated with an anatomy. The method may include generating a configuration associated with a physical anatomical model that may be representative of the virtual anatomical model. The method may include forming the physical anatomical model. The physical anatomical model may include a body associated with an anatomical profile of a bone. At least a first portion of the body may include a first material that may be transparent or translucent for viewing an interior portion of the body.

These and other features may be best understood from the following specification and drawings, the following of which is a brief description.

### BRIEF DESCRIPTION OF THE DRAWINGS

FIG. 1 discloses a planning system according to an implementation.

FIG. 2 discloses another implementation of a planning system including a user interface.

FIG. 3 discloses the user interface of FIG. 2 including a display window including various parameters.

FIG. 4 discloses the user interface of FIG. 2 including display windows depicting a virtual anatomical model.

FIGS. 5-7 disclose the user interface of FIG. 2 including display windows depicting aspects of the virtual anatomical model of FIG. 4.

FIG. 8 discloses the user interface of FIG. 2 including display windows depicting a fracture pattern relative to the virtual anatomical model of FIG. 7.

FIGS. 9A-9D disclose aspects of the fracture pattern of FIG. 8.

FIG. 10 discloses the user interface of FIG. 2 including display windows depicting the fracture pattern relative to the virtual anatomical model of FIG. 8.

FIG. 11 discloses the user interface of FIG. 2 including display windows depicting a fracture volume relative to the virtual anatomical model of FIG. 8.

FIG. 12A discloses a sectional view of aspects of the fracture volume of FIG. 11.

FIG. 12B discloses an isolated view of the fracture volume of FIG. 11.

FIG. 13 discloses a sectional view of a physical anatomical model according to an implementation.

FIG. 14 discloses virtual anatomical models including respective fracture volumes according to an implementation.

FIG. 15 discloses the fracture volumes of FIG. 14 with the virtual anatomical models in phantom.

FIGS. 16-17 disclose isolated views of the fracture volumes of FIG. 14 at different orientations.

FIG. 18 discloses a sectional view of one of the virtual anatomical models and the associated fracture volume of FIG. 14.

FIG. 19 discloses a system including a physical anatomical model according to another implementation.

FIG. 20 discloses selected portions of the physical anatomical model of FIG. 19.

FIGS. 21 and 22 disclose an implant within the physical anatomical model of FIGS. 19 and 20.

FIG. 23 discloses a method of planning and implementing a surgical procedure utilizing physical anatomical model(s).

FIG. 24 disclose a technique for forming a physical anatomical model.

Like reference numbers and designations in the various drawings indicate like elements.

### DETAILED DESCRIPTION

This disclosure relates to surgical systems, devices and methods for planning and implementing surgical procedures utilizing physical models of anatomy. Physical anatomical models may be utilized to rehearse and train for various surgical procedures, including the repair of fractures and other deformities.

The disclosed techniques may be utilized to provide the surgeon a preoperative planning and/or training experience that may be targeted or tailored to the surgeon based on procedure, skill set, experience, etc. The surgeon may select a particular configuration of a virtual anatomical model that may be fabricated or otherwise formed to establish a physical anatomical model based on the anatomy or pathology that the surgeon may intend to treat. The surgeon may utilize the physical anatomical model to train with particular instrumentation, implants and other devices that may be intended for a planned surgery to treat a patient.

The surgeon, assistant or other user may interact with a graphical user interface (GUI) to select various parameters or characteristics of the physical anatomical model. The parameters may include anatomy, patient, fracture classification, case, etc., to establish a desired configuration of the physical anatomical model. The surgeon may tailor or select one or more variables or parameters specific to a fracture classification scheme, depending on what the surgeon would like to train. The specified parameters may be represented in the physical anatomical model.

Various techniques may be utilized to establish the physical anatomical models, including any of the techniques disclosed herein. A virtual fracture pattern (e.g., virtual fracture path) and/or virtual fracture volume may be established relative to a virtual anatomical model. The fracture pattern may be established based on one or more parameters of a fracture classification scheme. The virtual fracture volume may be established along or otherwise adjacent to the fracture pattern. A physical fracture path and/or physical fracture volume may be established based on the virtual fracture pattern and/or virtual fracture volume, which may be incorporated in the physical anatomical model. The fracture volume may establish a relatively weaker localized region of the physical anatomical model, which may facilitate fragmentation of the physical anatomical model. The surgeon may reassemble (e.g., reduce) the fragment(s) of the physical anatomical model. The surgeon may secure the fragment(s) to each other and/or a remainder of the physical anatomical model, such as with a bone plate or another implant.

The physical anatomical model may include one or more portions that may incorporate a transparent or translucent material. The portion(s) may be utilized to view an interior of the physical anatomical model. In implementations, the transparent or translucent material may establish a viewing portal to the interior of the physical anatomical model. A light source may be utilized to illuminate the interior. Adjacent portion(s) of the physical anatomical model may be relatively more opaque than at least the material establishing the viewing portal. The adjacent portions may substantially surround a perimeter of the viewing portal.

The repaired physical anatomical model may serve as an artifact for the surgeon. The surgeon may leave a training facility with a revised physical anatomical model once training is completed. The surgeon may refer to the revised physical anatomical model prior to and during a surgical procedure on a respective patient.

A system for an orthopaedic procedure according to an implementation may include a physical anatomical model including a main body representative of anatomy. The main body may include a bone component. The bone component may include an exterior having a surface contour representative of a bone. The bone component may include a first portion establishing the exterior and a first material. The bone component may include a second portion adjacent to the first portion. The second portion may include a second material that may differ from the first material. The second material may transparent or translucent for viewing an interior of the bone component.

In any implementations, the second material may be transparent.

In any implementations, the second material may be translucent.

In any implementations, the second material may establish a viewing portal to the interior of the bone component. The interior of the bone component may include an internal cavity that may be at least partially aligned with the viewing portal. The internal cavity may be dimensioned to at least partially receive an orthopaedic implant.

In any implementations, the internal cavity may extend at least partially through the first portion of the bone component.

In any implementations, the internal cavity may include a deployment region dimensioned to at least partially receive a portion of the orthopaedic implant, which may be movable between an undeployed state and a deployed state. The deployment region may be at least partially aligned with the viewing portal.

In any implementations, the deployment region may include a conical void associated with a geometry of the portion of the implant in the deployed state.

In any implementations, the bone may be a fibula.

In any implementations, the second portion may be adjacent to an intermediate portion and/or a proximal portion of the bone component.

In any implementations, the first material may have a transparency that may be less than a transparency of the second material.

In any implementations, the first material may be substantially opaque.

In any implementations, the first portion may be distal to the second portion.

In any implementations, the first portion may include a joint region of the bone component. The joint region may be representative of an orthopaedic joint.

In any implementations, a light source may be at least partially embedded in the main body. The light source may be operable to illuminate the interior.

In any implementations, the light source may be at least partially embedded in the bone component.

In any implementations, the second material may establish a viewing portal to the interior of the bone component. An opaque material may surround a perimeter of the viewing portal.

In any implementations, a soft tissue component may be on the bone component. The soft tissue component may be representative of soft tissue.

In any implementations, the bone component may include a fracture path that may establish one or more localized regions. The bone component may be severable along the fracture path to establish one or more fragments that may be associated with a respective one of the one or more localized regions.

In any implementations, the interior of the bone component may include an internal cavity that may extend through the fracture path and may be at least partially aligned with a viewing portal established by the second material. The internal cavity may be dimensioned to at least partially receive an orthopaedic implant.

A system for a surgical procedure according to an implementation may include a physical anatomical model including a body associated with an anatomical profile of a bone. At least a first portion of the body may be formed of a first material that may be transparent or translucent, through which an interior of the body may be viewed. A light source may be operable to illuminate the interior of the body.

In any implementations, the light source may be at least partially embedded in the body.

In any implementations, the bone may be a long bone.

A method of rehearsing for a surgical procedure according to an implementation may include accessing a virtual anatomical model associated with an anatomy. The method may include generating a configuration associated with a physical anatomical model that may be representative of the virtual anatomical model. The method may include forming the physical anatomical model. The physical anatomical model may include a body associated with an anatomical profile of a bone. At least a first portion of the body may include a first material that may be transparent or translucent for viewing an interior portion of the body.

In any implementations, the body may include an exterior having a surface contour representative of a cortical wall of the bone. The body may include a second portion that may establish the exterior adjacent to the first portion. The second portion may include a second material having an opacity that may be greater than an opacity of the first material.

In any implementations, the method may include illuminating the interior portion.

In any implementations, the illuminating step may include illuminating the interior portion with a light source that may be at least partially embedded in the body.

In any implementations, the forming step may include printing layers of material on each other to establish the physical anatomical model.

In any implementations, the method may include forming an internal cavity in the interior portion.

In any implementations, the forming step may include at least partially filling the internal cavity with a material having at least one characteristic that may differ from the first material.

In any implementations, the internal cavity may include a deployment region adjacent to the first portion that may be dimensioned to receive a portion of an orthopaedic implant, which may have an undeployed state and a deployed state.

In any implementations, the first portion may be at least partially aligned with the deployment region such that the deployment region may be visible from outside of the physical anatomical model.

In any implementations, the method may include inserting the orthopaedic implant at least partially into the internal cavity.

In any implementations, the method may include deploying the implant within the deployment region.

In any implementations, the method may include illuminating the first material when the implant is in the deployed state.

In any implementations, the bone may be a long bone.

In any implementations, the first portion may include a viewing portal for viewing the interior portion from outside of the physical anatomical model.

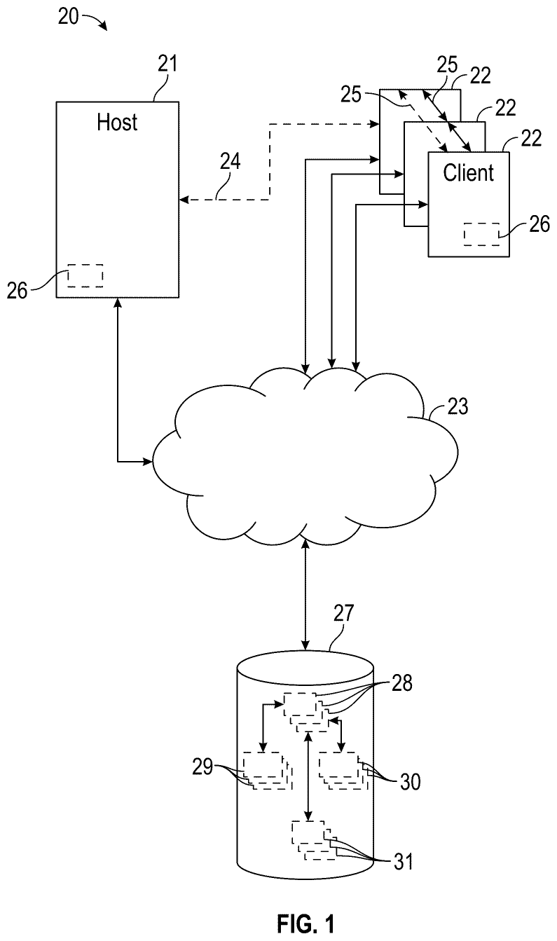

FIG. 1 discloses a planning system 20 that may be utilized for planning surgical procedures according to an implementation. The system 20 may be used for planning orthopaedic procedures, including pre-operatively, intra-operatively and/or post-operatively to create, edit, execute and/or review surgical plans. The system 20 may be used for training and rehearsing for various surgical procedures, including prior cases and surgical plans for patients and hypothetical cases.

The system 20 may include a host computer 21 and one or more client computers 22. The host computer 21 may be configured to execute one or more software programs. In implementations, the host computer 21 may include more than one computer jointly configured to process software instructions serially or in parallel.

The host computer 21 may be operable to communicate with one or more networks such as a network 23 comprised of one or more computing devices. The network 23 may be a private local area network (LAN), a private wide area network (WAN), the Internet, or a mesh network.

The host computer 21 and each client computer 22 may include one or more of a computer processor, memory, storage means, network device and input and/or output devices and/or interfaces. The input devices may include a keyboard, mouse, etc. The output device may include a monitor, speakers, printers, etc. The memory may include UVPROM, EEPROM, FLASH, RAM, ROM, DVD, CD, a hard drive, or other computer readable medium which may store data and/or other information relating to the features and techniques disclosed herein. The host computer 21 and each client computer 22 may be a desktop computer, laptop computer, smart phone, tablet, or any other computing device. The interface may facilitate communication with the other systems and/or components of the network 23.

Each client computer 22 may be configured to communicate with the host computer 21 directly via a direct client interface 24 or over the network 23. The client computers 22 may be configured to execute one or more software programs, such as various surgical tools. Each client computer 22 may be operable to access and locally and/or remotely execute a planning environment 26. The planning environment 26 may be a standalone software package or may be incorporated into another surgical tool. The planning environment 26 may be configured to communicate with the host computer 21 either over the network 23 or directly through the direct client interface 24. In implementations, the client computers 22 may be configured to communicate with each other directly via a peer-to-peer interface 25.

The planning environment 26 may provide a display or visualization of one or more virtual anatomical models 29 and related images and/or one or more implant models 30 via one or more graphical user interfaces (GUI). Each anatomical model 29, implant model 30, and related images and other information may be stored in one or more files or records according to a specified data structure.

The system 20 may include at least one storage system 27, which may be operable to store or otherwise provide data to other computing devices. The storage system 27 may be a storage area network device (SAN) configured to communicate with the host computer 21 and/or the client computers 22 over the network 23. In implementations, the storage system 27 may be incorporated within or directly coupled to the host computer 21 and/or client computers 22. The storage system 27 may be configured to store one or more of computer software instructions, data, database files, configuration information, etc.

In implementations, the system 20 may be a client-server architecture configured to execute computer software on the host computer 21, which may be accessible by the client computers 22 using either a thin client application or a web browser executing on the client computers 22. The host computer 21 may load the computer software instructions from local storage, or from the storage system 27, into memory and may execute the computer software using the one or more computer processors.

The system 20 may include one or more databases 28. The databases 28 may be stored at a central location, such as the storage system 27. In implementations, one or more databases 28 may be stored at the host computer 21 and/or may be a distributed database provided by one or more of the client computers 22. Each database 28 may be a relational database configured to associate one or more anatomical models 29 and/or one or more implant models 30 to each other and/or a surgical plan 31. Each surgical plan 31 may be associated with a respective patient. Each anatomical model 29, implant model 30 and surgical plan 31 may be assigned a unique identifier or database entry. The database 28 may be configured to store data corresponding to the anatomical models 29, implant models 30 and surgical plans 31 in one or more database records or entries, and/or may be configured to link or otherwise associate one or more files corresponding to each respective anatomical model 29, implant model 30 and surgical plan 31. Anatomical models 29 stored in the database(s) 28 may correspond to respective patient anatomies from prior and/or planned surgical cases, and may be arranged into one or more predefined categories such as sex, age, ethnicity, size, defect category, procedure type, etc. The anatomical models 29 and/or implant models 30 may be associated with respective instrumentation and devices to implement the associated surgical plan 31.

Each anatomical model 29 may include information obtained from one or more medical devices or tools, such as a computerized tomography (CT), magnetic resonance imaging (MRI) machine and/or X-ray machine, that may obtain one or more images of a patient. The anatomical model 29 may include one or more digital images and/or coordinate information relating to an anatomy of the patient obtained or derived from the medical device(s). In implementations, one or more of the anatomical models 29 may be created by a designer and may represent a hypothetical anatomy. Each implant model 30 may include coordinate information associated with a predefined design. The planning environment 26 may incorporate and/or interface with one or more modeling packages, such as a computer aided design (CAD) package, to render the models 29, 30 as two-dimensional (2D) and/or three-dimensional (3D) volumes or constructs. Each anatomical model 29 and implant model 30 may correspond to 2D and/or 3D geometry, and may be utilized to generate a wireframe, mesh and/or solid construct in a display.

The implant models 30 may correspond to implants and components of various configurations, shapes, sizes, procedures, instrumentation, etc. Each implant may include one or more components that may be situated at a surgical site including plates, anchors, screws, nails, suture, grafts, etc. Each implant model 30 may correspond to a single component or may include two or more components that may be configured to establish an assembly. The implant models 30 may include base plates coupled to an articulation member, bone plates configured to interconnect adjacent bones or bone fragments, intermedullary nails, suture anchors, etc. The articulation member may have an articular surface dimensioned to mate with an articular surface of an opposed bone or implant.

Each surgical plan 31 may be associated with one or more of the anatomical models 29 and/or implant models 30. The surgical plan 31 may include one or more revisions to the anatomical model 29 and information relating to a position of an implant model 30 relative to the original and/or revised anatomical model 29. The surgical plan 31 may include coordinate information relating to the revised anatomical model 29 and a relative position of the implant model 30 in predefined data structure(s). Revisions to each anatomical model 29, implant model 30 and surgical plan 31 may be stored in the database 28 automatically and/or in response to user interaction with the system 20.

One or more surgeons, assistants and other clinical users may be provided with a planning environment 26 via the client computers 22 and may simultaneously access each anatomical model 29, implant model 30 and surgical plan 31 stored in the database(s) 28. Each user may interact with the planning environment 26 to create, view and/or modify various aspects of the surgical plan 31. Each client computer 22 may be configured to store local instances of the anatomical models 29, implant models 30 and/or surgical plans 31, which may be synchronized in real-time or periodically with the database(s) 28. The planning environment 26 may be a standalone software package executed on a client computer 22 or may be provided as one or more services executed on the host computer 21.

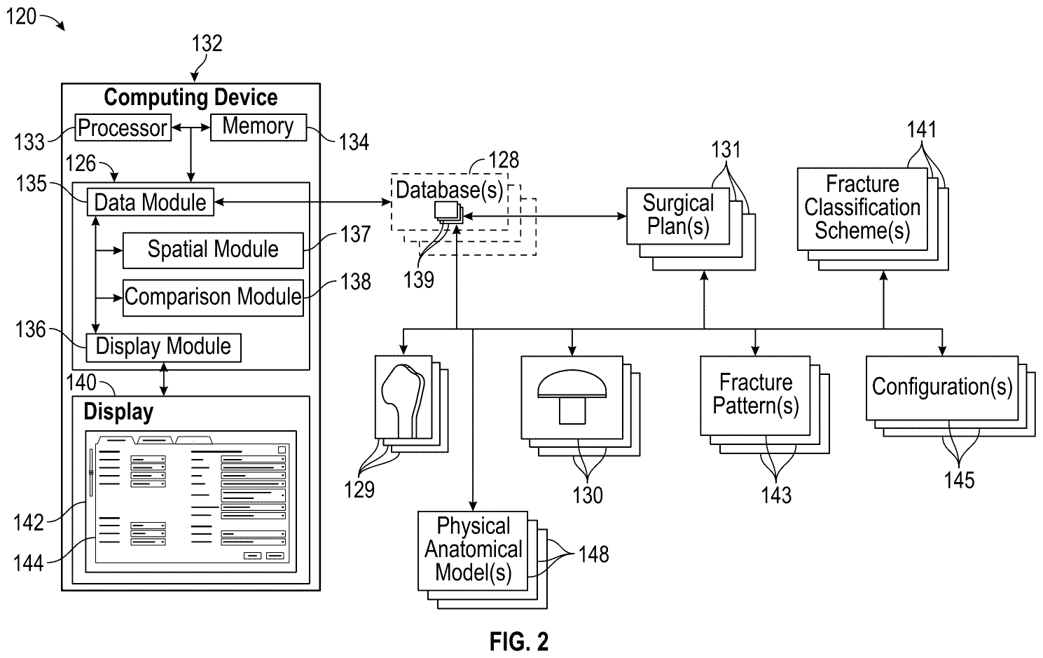

FIG. 2 discloses a surgical system 120 according to an implementation. The system 120 be utilized to facilitate planning, rehearsing and/or training for a surgical procedure. The system 120 may be utilized to plan, rehearse, train and implement various orthopaedic and other surgical procedures, such as an arthroplasty to repair a joint. The system 120 may be utilized in planning a resection or revision of one or more bones. The system 120 may be utilized in planning placement of an implant to restore functionality to a bone and/or joint. The system 120 may be utilized in planning the repair of a fracture of one or more bones, including one or more long bones such as a fibula, tibia, femur or humerus. The planning system 120 may be utilized in the repair of locations of the patient and other surgical procedures including repair of joints such as an ankle, wrist, hand, hip or knee, shoulder and including repair of other tissue such as cartilage, muscles, tendons and ligaments.

The system 120 may be configured to generate one or more physical anatomical models, including any of the physical anatomical models disclosed herein. The surgeon may perform one or more modifications to the physical anatomical model or otherwise interact with the physical anatomical model to rehearse or train for a surgical procedure. The system 120 may be configured to generate configuration(s) associated with respective physical anatomical model(s). The configuration may be utilized in the formation of a physical anatomical model. Each physical anatomical model may be representative of a virtual anatomical model 129, including a substantially or generally corresponding geometry, texture density, porosity, color, opacity, etc. as the virtual anatomical model 129. The virtual anatomical model 129 may be associated with an anatomy, such as the anatomy of a patient and/or a hypothetical anatomy. The anatomical models 129 may include one or more anatomical features. The anatomical features may be representative of anatomy, including one or more bones including cartilage, cortical and/or cancellous bone tissue, soft tissue including muscle, ligaments and/or tendons, etc., and/or other tissue.

The system 120 may include a computing device 132. The computing device 132 may include one or more processors 133 coupled to memory 134. The computing device 132 may include any of the computing devices disclosed herein, such as the host computer 21 and/or client computer 22 of FIG. 1. The processor(s) 133 may be configured to individually and/or collectively execute a planning environment 126 for creating, editing, executing and/or reviewing one or more surgical (e.g., pre-operative) plans 131 during pre-operative, intra-operative and/or post-operative phases of a surgical procedure. The processor 133 may be configured to access one or more virtual anatomical models 129 from a storage location such as the memory 134. The anatomical model 129 and surgical plan 131 may be associated with an actual case for a patient or may be a hypothetical case established for rehearsal and/or training surgeons, assistants, medical staff and other clinical users.

The planning environment 126 may include at least a data module 135, display module 136, spatial module 137 and comparison module 138. The processor(s) 133 may be configured to individually and/or collectively execute the data module 135, display module 136, spatial module 137 and comparison module 138. Although four modules are disclosed in the implementation of FIG. 2, it should be understood that fewer or more than four modules may be utilized and/or one or more of the modules may be combined to provide the disclosed functionality.

The data module 135 may be configured to access, retrieve and/or store data and other information in the database(s) 128 corresponding to one or more virtual anatomical model(s) 129, implant model(s) 130 and/or surgical plan(s) 131. The data and other information may be stored in the database 128 as one or more records or entries 139. In implementations, the data and other information may be stored in one or more files that may be accessible by referencing one or more objects or memory locations referenced by the records 139.

The memory 134 may be configured to access, load, edit and/or store instances of one or more anatomical models 129, implant models 130 and/or surgical plans 131 in response to one or more commands from the data module 135. The data module 135 may be configured to cause the memory 134 to store a local instance of the anatomical model(s) 129, implant model(s) 130 and/or surgical plan(s) 131 which may be synchronized with records 139 in the database(s) 128.

The display module 136 may be configured to display data and other information relating to one or more surgical plans 131 in at least one graphical user interface (GUI) 142. The computing device 132 may be coupled to a display device 140. The display module 136 may be configured to cause the display device 140 to display the virtual anatomical model 129 in the user interface 142. A surgeon or other clinical user may interact with the user interface 142 via the planning environment 126 to create, edit and/or review aspects of one or more anatomical models 129. The surgeon or other user may interact with the user interface 142 via the planning environment 126 to create, edit, execute and/or review aspects of one or more surgical plans 131.

Each surgical plan 131 may be associated with one or more (e.g., original) virtual anatomical models 129 prior to any revisions, which may substantially or generally approximate an anatomy. Each surgical plan 131 may be associated with one or more (e.g., revised) virtual anatomical models 129 that may incorporate one or more revisions to the anatomy and/or an associated physical anatomical model. The original and revised anatomical models 129 may be associated with each other in the surgical plan 131. In implementations, the revisions may be stored as one or more parameters of the original anatomical model 129.

The planning system 120 may be configured to generate a link to a surgical plan 131. The surgeon, assistant or other clinical user may interact with the link to review and edit the surgical plan 131. Interacting with the link may cause the planning system 120 to display or otherwise present aspects of the surgical plan 131 in the graphical user interface 142.

The planning system 120 may be utilized to generate a physical instance of a virtual anatomical model 129 that a surgeon may utilize for rehearsing or training for an orthopaedic procedure, such as the repair of a fracture. The surgeon may interact with a fractured state of a physical anatomical model, which may be associated with the virtual anatomical model 129. Each fracture may be classified according to one or more fracture classification schemes 141. Various fracture classification schemes may be utilized in accordance with the teachings disclosed herein, including predefined industry classification schemes and/or user-defined classification schemes. Industry defined classification schemes may include the Müller AO Classification of fractures, the Neer Classification, and the AO Foundation and Orthopaedic Trauma Association (AO/OTA) Fracture Classification Scheme. The AO/OTA Fracture Classification Scheme may include a 2018 revision of the AO/OTA Fracture and Dislocation Classification Compendium released by the AO Foundation. Other fracture classification schemes may be utilized in accordance with the teachings disclosed herein, including any known classification scheme recognized in the medical community.

The planning system 120 may be adapted to access one or more fracture classification schemes 141. The comparison module 138 may be adapted to access one or more fracture patterns (e.g., virtual fracture path) 143. Various techniques may be utilized to establish the fracture pattern 143, including any of the techniques disclosed herein. The planning system 120 may be adapted to associate each fracture patterns 143 with one or more of the fracture classification schemes 141. The data module 135 may be configured to access, retrieve and/or store data and other information in the database(s) 128 corresponding to one or more fracture classification schemes 141 and/or fracture patterns 143. The fracture classification schemes 141 and/or fracture pattern 143 may be predefined and/or may be established by the comparison module 138. In implementations, the planning system 120 may generate one or more fracture classification schemes 141 and/or fracture patterns 143 automatically and/or in response to user input. The fracture patterns 143 may be generated utilized various techniques, such as finite element analysis (FEA) and other parametric modeling.

The comparison module 138 may be adapted to associate each anatomical model 129 with one or more fracture classification schemes 141 and/or fracture patterns 143. The comparison module 138 may be adapted to assign one or more fracture classification schemes 141 to each fracture pattern 143, either automatically and/or in response to user interaction with the user interface 142 and/or another portion of the planning system 120. The data module 135 may be adapted to store and/or access an instance of each anatomical model 129 and an associated fracture classification scheme 141 and/or fracture pattern 143 in the database(s) 128 or another memory location. The comparison module 138 may be adapted to generate, revise or otherwise associate a surgical plan 131 with an anatomical model 129, fracture classification scheme 141 and/or fracture pattern 143.

Each fracture classification scheme 141 and/or fracture pattern 143 may be stored in a respective predefined data structure(s) in the database 128 or another portion of the system 120. The data and other information associated with the respective fracture classification scheme 141 and/or fracture pattern 143 may be stored in the database 128 as one or more respective records or entries 139. In implementations, the data and other information may be stored in one or more files that may be accessible by referencing one or more objects or memory locations referenced by the records 139. The memory 134 may be configured to access, load, edit and/or store instances of one or more fracture classification schemes 141 and/or fracture patterns 143 in response to one or more commands from the data module 135. The data module 135 may be configured to cause the memory 134 to store a local instance of the fracture classification scheme(s) 141 and/or fracture pattern(s) 143, which may be synchronized with records 139 in the database(s) 128.

The planning system 120 may be utilized to establish one or more physical anatomical models 148, including any of the physical anatomical models disclosed herein. The physical anatomical model 148 may be representative of an associated virtual anatomical model 129.

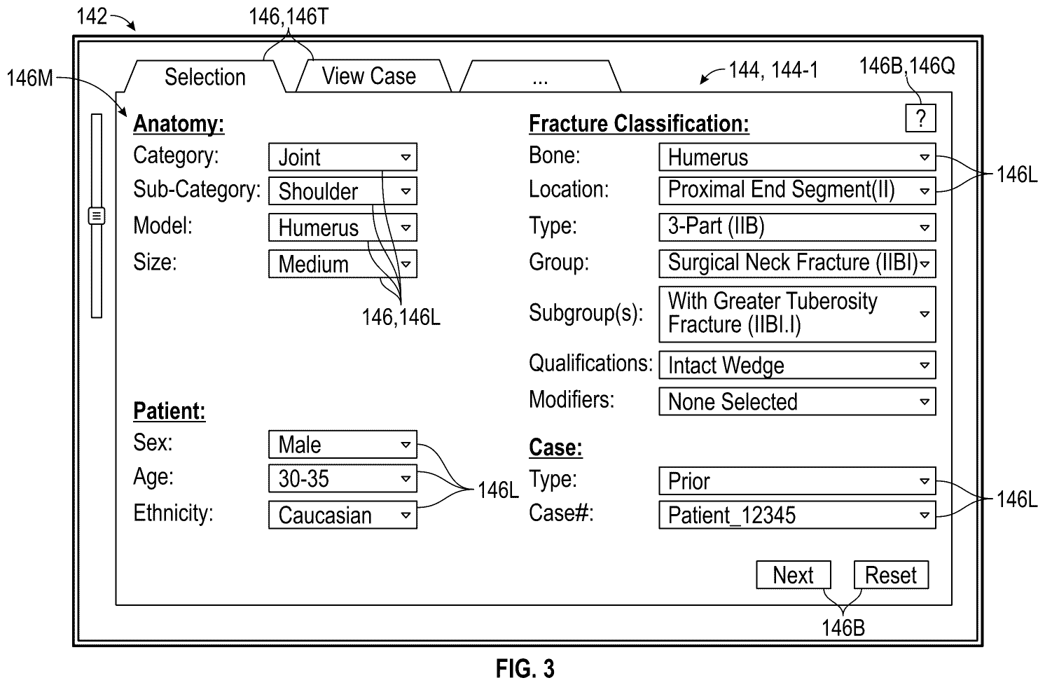

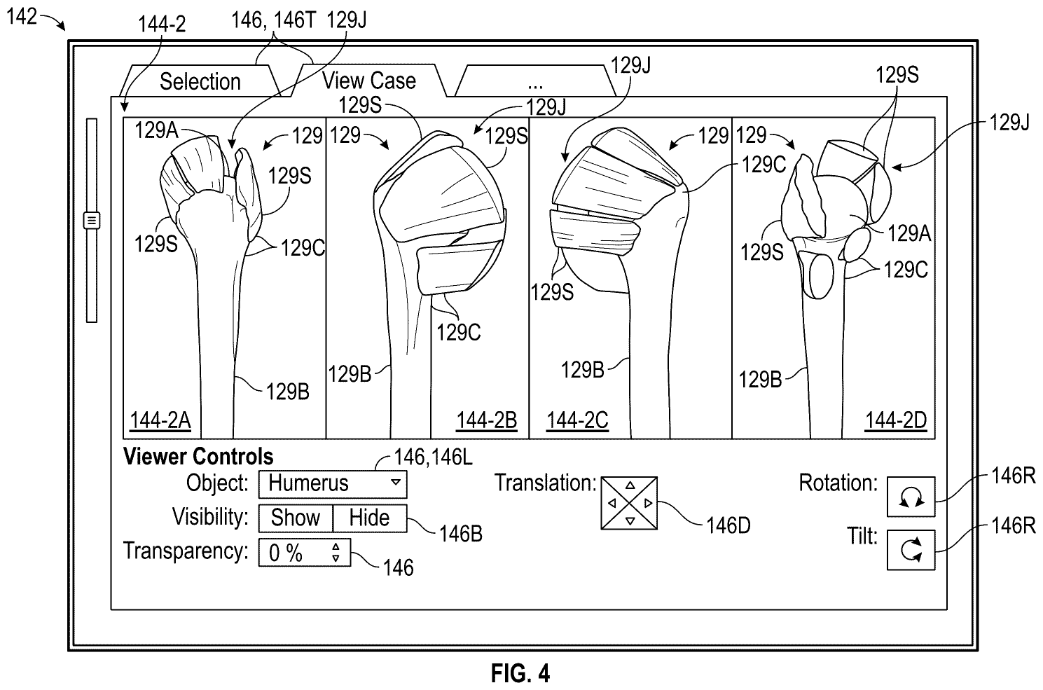

Referring to FIG. 3, with continuing reference to FIG. 2, the user interface 142 may include one or more display windows 144 and one or more objects 146, such as a first display window 144-1. The objects 146 may include graphics such as menus, tabs, lists, entry fields and buttons accessible by user interaction, such as tabs 146T, buttons 146B, drop-down lists 146L, menus 146M, directional indicators 146D, 146R (e.g., FIG. 4), and graphics associated with respective display window(s) 144. In implementations, one or more entries may be specified in respective entry fields, including any parameters associated with the lists 146L. Geometric objects, including selected virtual anatomical model(s) 129, implant model(s) 130, fracture pattern(s) 143 and/or other information relating to a surgical plan 131 may be displayed in one or more of the display windows 144.

The comparison module 138 may be configured to assign a fracture pattern 143 to the virtual anatomical model 129 based on one or more parameters, including any of the parameters disclosed herein. The parameters may be associated with a predefined fracture classification scheme 141. The comparison module 138 may be configured to assign the fracture pattern 143 to the virtual anatomical model 129 in response to setting one or more parameters associated with the fracture classification scheme 141.

The surgeon or clinical user may interact with the display window 144 and/or another portion of the user interface 142 to select one or more anatomical models 129. Various parameters may be utilized to select the anatomical model(s) 129. The anatomical models 129 may be categorized by anatomy, patient, defect (e.g., fracture classification), case, etc. The parameters may be associated with respective objects 146 of the user interface 142. The parameters of the display window 144 may be interconnected to provide a filtering feature such that each selection of a parameter may cause the remaining parameter(s) to be filtered to depict available options. Each parameter may be associated with a set of anatomical models 129 accessible by the planning environment 126.

The display module 136 may be adapted to present one or more parameters associated with the anatomy, patient, fracture classification scheme and/or case to the surgeon or clinical user in the display window 144. The surgeon or clinical user may interact with the user interface 142 to select or otherwise specify one or more of the parameters. Anatomical parameters may be arranged in one or more lists 146L by category (e.g., joint, etc.), sub-category (e.g., shoulder, ankle, hip, hand, foot, etc.), model (e.g., glenoid, humerus, femur, pelvis, tibia, fibula, etc.) and anatomical size (e.g., small, medium, large). The categories may be subdivided by gross anatomy including surface anatomy (e.g., the external body), regional anatomy (e.g., specific regions of the body), and systemic anatomy (e.g., specific organ systems). The data module 135 may be adapted to cause the display module 136 to populate entries associated with the virtual anatomical model(s) 129 and other parameters including category, sub-category, model and/or size in respective lists 146L. The spatial module 137 may be configured to scale a geometry of the selected anatomical model 129 in response to selection of an anatomical size. The surgeon or clinical user may select or otherwise specify the anatomical parameters including category, sub-category, model and/or size of the anatomy in response to interaction with the display window 144 and/or another portion of the user interface 142. Each list 146L may be associated with one or more virtual anatomical model(s) 129. The anatomical model 129 may be associated with an anatomy of a patient, such as a prior case or a planned case, and/or a hypothetical anatomy. The surgeon or clinical user may select or otherwise specify parameter(s) associated with respective virtual anatomical model(s) 129.

The anatomical models 129 may be categorized by patient parameters. Various patient parameters may be utilized, such as sex, age and ethnicity. The patient parameters may be presented in respective lists 146L. The data module 135 may be adapted to cause the display module 136 to populate one or more patient parameters associated in the respective lists 146L. The data module 135 may be adapted to cause the display module 136 to populate entries associated with the anatomy and other parameters including category, sub-category, model and/or size in the respective lists 146L in response to specifying parameters associated with the patient population.

Case parameters may include case type (e.g., prior, planned and hypothetical), case number, etc. The surgeon may interact with the list(s) 146L and/or another portion of the user interface 142 to select and/or review a particular case, such as a prior, planned or hypothetical case associated with a surgical plan 131, which may be filtered by the data module 135 based on previous selection(s) of the parameters. The surgeon may interact with the user interface 142 to review prior cases, including prior cases for a particular surgical procedure, anatomy and/or group of patients. The planning system 120 may be configured to provide analysis of the prior case such as biometric testing of a repaired joint, finite element analysis (FEA), etc. The surgeon or clinical user may select a virtual anatomical model 129 corresponding to an intended patient. The selected virtual anatomical model 129 may correspond to an acquired CT scan of the patient. The surgeon may select a virtual anatomical model 129 that may be associated with a particular classification.

The data module 135 may be adapted to cause the display module 136 to populate entries associated with a case, such as type (e.g., prior, planned or hypothetical) and/or case number in respective lists 146L. The data module 135 may be adapted to cause the display module 136 to populate entries associated with the case and other parameters including type and/or case number in the respective lists 146L in response to specifying parameters associated with the patient population.

The surgical plan 131 may be associated with an anatomical model 129 prior to any revisions and may be associated with another (e.g., revised) anatomical model 129 incorporating one or more revisions based on implementation of an associated surgical procedure. Revisions may include removal of material utilizing one or more drilling, milling, resection, reaming and cutting operations. Revisions may include one or more fragmentary states of the anatomical model 129, including prior to and/or subsequent to registration of any associated fragments.

The display module 136 may be adapted to present one or more parameters of a fracture classification scheme 141 associated with the virtual anatomical model 129 in the user interface 142. The display module 136 may be adapted to display the parameter(s) associated with the classification scheme 141 in the first display window 144-1. The display module 136 may be configured to present one or more parameters of a respective classification scheme 141 in response to selection of a bone type and/or fracture location. The display module 136 may be adapted to present one or more parameters associated with the classification scheme 141 in response to selection of a bone type (e.g., humerus, femur, tibia, fibula, etc.) and/or fracture location (e.g., proximal humeral fracture location, distal fibula fracture location, etc.) from one or more menus 146L and/or another portion of the user interface 142. The data module 135 may be adapted to cause the display module 136 to populate entries associated with parameter(s) of a fracture classification scheme 141 in respective lists 146L and/or other portion of the user interface 142. The fracture classification scheme 141 may be selected automatically and/or manually in response to one or more selections associated with anatomy, patient and/or case. The entries may include bone type (e.g., humerus or fibula), location (e.g., proximal or distal end segment), type (e.g., two-part, three-part or four-part), group (e.g., surgical neck fracture) and subgroup(s) (e.g., with greater tuberosity fracture) and other parameters of the associated fracture classification scheme 141 such as qualifier(s) and/or modifier(s) in respective lists 146L.

The data module 135 may be adapted to access a virtual anatomical model 129 from memory, such as the memory 134 and/or database 128, in response to selecting one or more parameters in the display window 144 of the graphical user interface 142. The data module 135 may be configured to select an anatomical model 129 from memory, such as the database 128 or memory 134, in response to user interaction with the display window 144 or another portion of the user interface 142. The data module 135 may select a fracture pattern 143 in response to one or more of the parameters of the fracture classification scheme 141 of the selected virtual anatomical model 129 being selected or otherwise specified.

The surgeon or clinical user may select the virtual anatomical model 129 according to a severity of various defects, such as mild, severe, non-pathological, fractures, etc. Defect parameters may be established for the various defects and may be arranged by classification, subclassification, etc. The surgeon, assistant or other user may interact with a button 146B (see, e.g., question mark button 146Q) for an explanation of the defect parameters. In implementations, selection of a virtual anatomical model 129 from the list 146L may cause a help screen to be generated and displayed with one or more fracture classification options in response to selection of the button 146Q. The fracture classification options may be associated with a respective fracture classification scheme 141, including any of the fracture classification schemes disclosed herein. The surgeon or clinical user may select from the various classification parameters to rehearse and/or train for a surgical procedure, including the treatment of a fractured bone.

Referring to FIG. 4, with continuing reference to FIG. 2, a selected virtual anatomical model 129 may be displayed in one or more display windows 144 of the user interface 142. In the implementation of FIG. 4, the various views of the virtual anatomical model 129 may be displayed in display windows 144-2, such as a second set of display windows 144-2A through 144-2D. Each virtual anatomical model 129 may include one or more components 129C. The components 129C may include various representations of tissue, such as bone and soft tissue. Bone(s) may be represented by respective bone volume(s) 129B. Soft tissue(s) may be represented by respective soft tissue volume(s) 129S. Various representations of soft tissue may be utilized, such as tendons, ligaments, musculature and other soft tissue. The anatomical model 129 may establish a portion of a joint 129J. The bone volume(s) 129B may include at least articular surface 129A that may be dimensioned to cooperate with an adjacent articular surface to establish the joint 129J. Although four display windows 144-2A to 144-2D are shown in FIG. 4, it should be understood that fewer or more than four display windows 144 may be utilized in accordance with the teachings disclosed herein. The surgeon or clinical user may interact with one or more of the objects 146 to observe various aspects of the anatomical model 129. In implementations, the surgeon or clinical user may interact with list(s) 146L to select the respective component 129C (e.g., humerus, fibula, etc.).

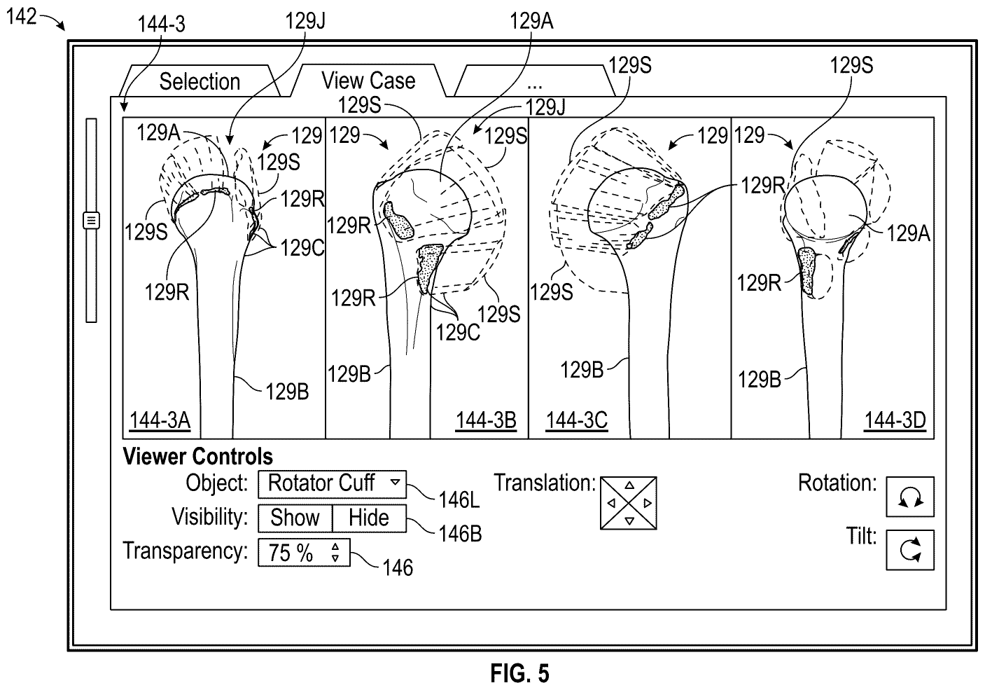

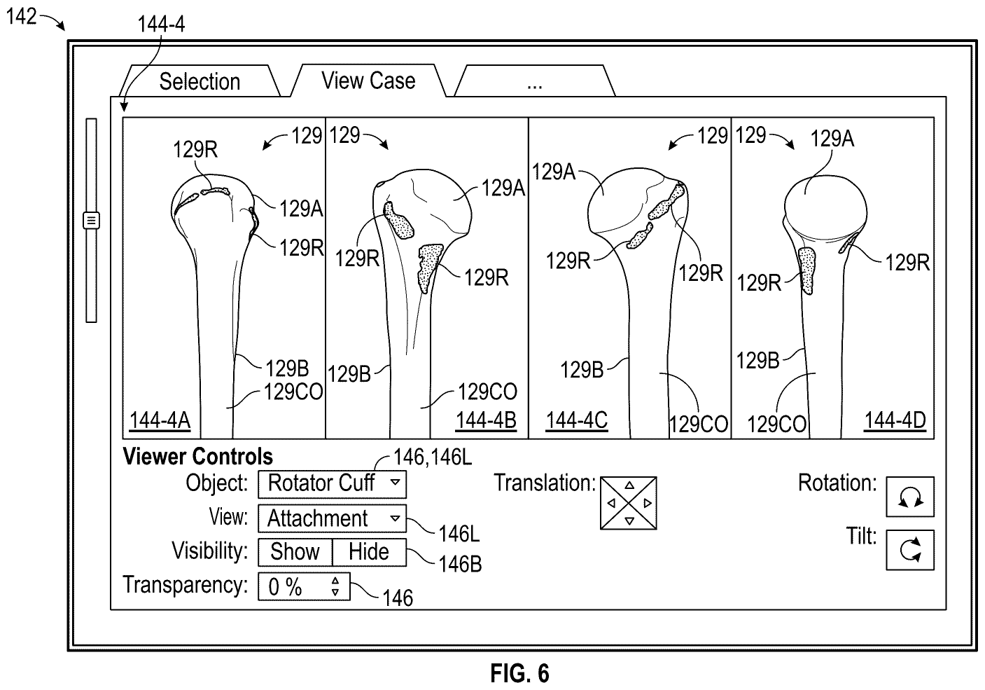

Referring to FIG. 5, with continuing reference to FIGS. 2 and 4, the surgeon or clinical user may interact with the user interface 142 to observe one or more aspects of the anatomical model 129. The user interface 142 may include one or more display windows 144-3, such as a third set of display windows 144-3A to 144-3D. The user may interact with a list 146L to select a specific portion of the soft tissue volume 129S, such as a rotator cuff. The display module 136 may be adapted to display one or more components 129C of the anatomical model 129, such as attachment regions 129R. The attachment regions 129R may be established along an interface between the bone volume 129B and the respective soft tissue volume 129S. The surgeon or clinical user may interact with one of the objects 146 to specify a transparency of one or more of these selected components 129C, such as one or more of the soft tissue volume 129S. Referring to FIG. 6, with continuing reference to FIGS. 2-3 and 5, the surgeon or clinical user may interact with the user interface 142 such as by interacting with one of the buttons 146B or another object 146 to select an attachment view within a list 146L such that the display module 136 may display a view of the attachment regions 129R with the soft tissue omitted (see, e.g., FIG. 5). The attachment regions 129R may be displayed in one or more display windows 144-4, such as a fourth set of display windows 144-4A to 144-4D.

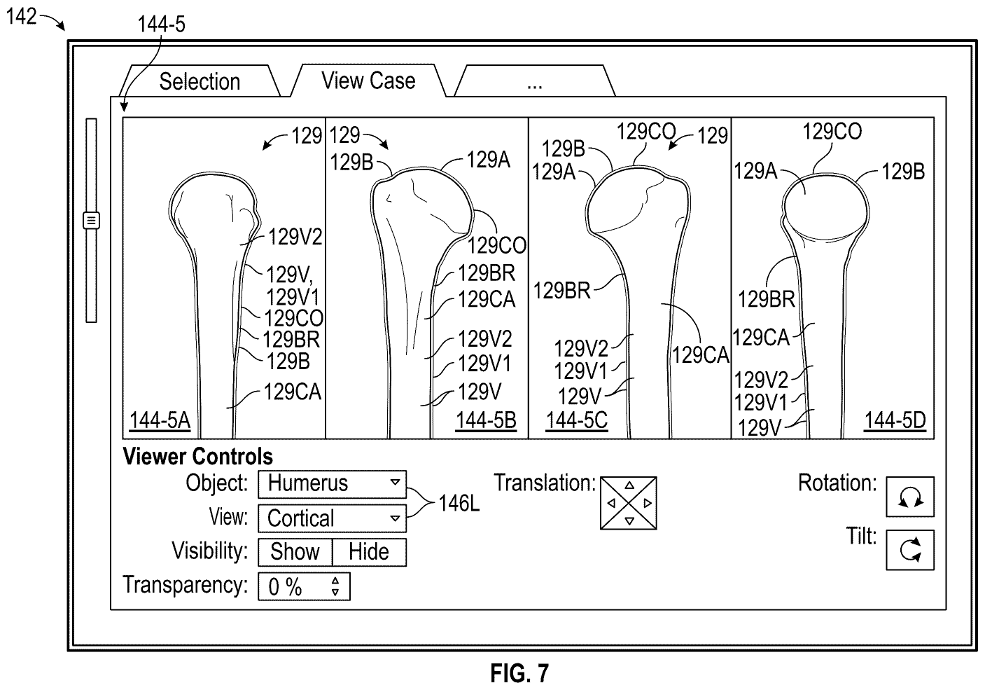

Referring to FIG. 7, with continuing reference to FIGS. 2, and 6, the surgeon or clinical user may interact with the user interface 142 to observe an isolated view of the bone volume 129B. The virtual anatomical model 129 may be displayed in one or more display windows 144-5, such as a set of fifth display windows 144-5A to 144-5D. The display module 136 may be adapted to display one or more aspects of the bone volume 129B, such as a cortical bone volume 129CO and/or cancellous bone volume 129CA. In implementations, the cortical bone volume 129CO may be displayed in phantom, and the cancellous bone volume 129CA may be displayed as a two-dimensional or three-dimensional solid.

The virtual anatomical model 129 may include one or more volumes 129V. One or more characteristics of the volumes 129V may be the same or may differ. The characteristics may include any of the characteristics disclosed herein, such as material composition and/or construction. In implementations, the volumes 129V may include a first volume 129V1 and a second volume 129V2. The first (e.g., cortical bone) volume 129V1 may be representative of cortical bone. The second (e.g., cancellous bone) volume 129V2 may be representative of cancellous bone. The cortical bone volume 129CO may establish the first volume 129V1. The cancellous bone volume 129CA may establish the second volume 129V2. The first volume 129V1 and the second volume 129V2 may include one or more characteristics that may be the same or may differ, such as material composition and/or construction. In implementations, the first and second volumes 129V1, 129V2 may differ in density. The different densities may be associated with different bone densities of an associated anatomy.

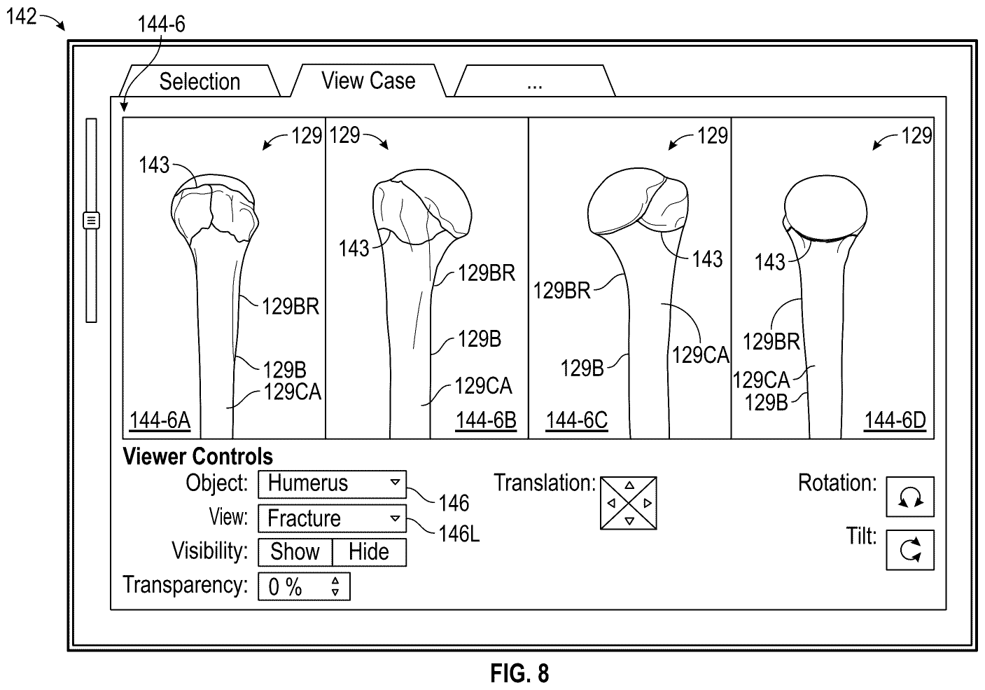

Referring to FIG. 8, with continuing reference to FIGS. 2 and 7, one or more fracture patterns 143 may be selected or assigned to each virtual anatomical model 129. In implementations, the data module 135 may be configured to access one or more fracture patterns 143 from the database(s) 128 and/or another data location internal and/or external to the planning system 120. The spatial module 137 may be configured to generate the fracture pattern 143.

The fracture pattern 143 may extend along a boundary region 129BR between the first volume 129V1 and the second volume 129V2 (see, e.g., FIG. 7). The boundary region 129BR may be established along an interface between the cortical bone volume 129CO and the cancellous bone volume 129CA (see, e.g., FIG. 7). The boundary region 129BR may follow along an external surface of the cancellous bone volume 129CA and/or an internal surface of the cortical bone volume 129CO.

The surgeon or clinical user may interact with the user interface 142 to observe one or more aspects of the selected or assigned fracture pattern 143. The spatial module 137 may be adapted to arrange the assigned or selected fracture pattern 143 relative to the respective bone volume 129B. In the implementation of FIG. 8, the display module 136 may be adapted to display one or more views of the bone volume 129B and the associated fracture pattern 143 in one or more display windows 144, such as a sixth set of display windows 144-6A to 144-D. The surgeon or clinical user may interact with the one or more objects such as a list 146L to select a fracture view.

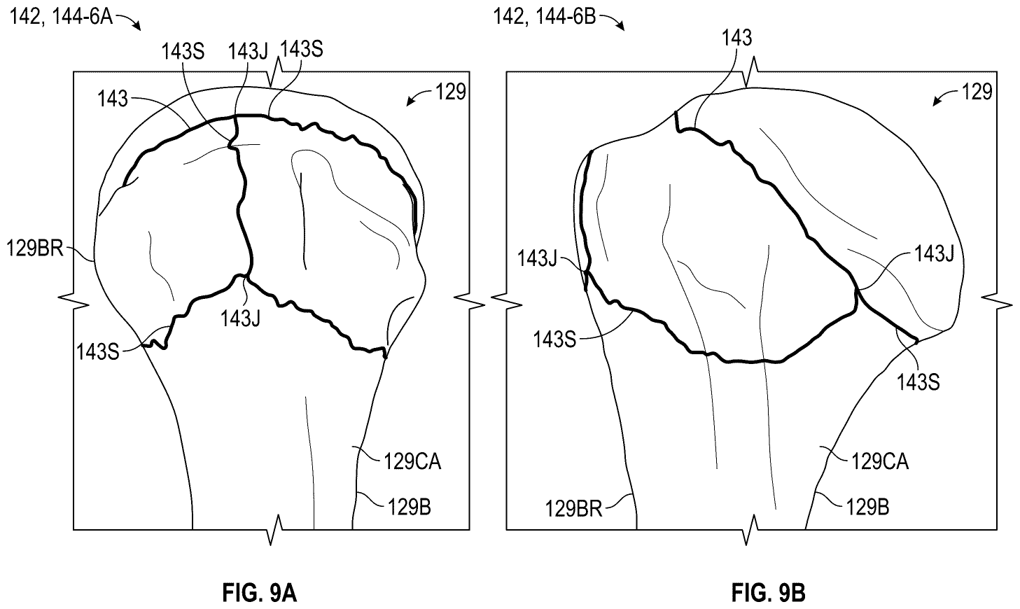

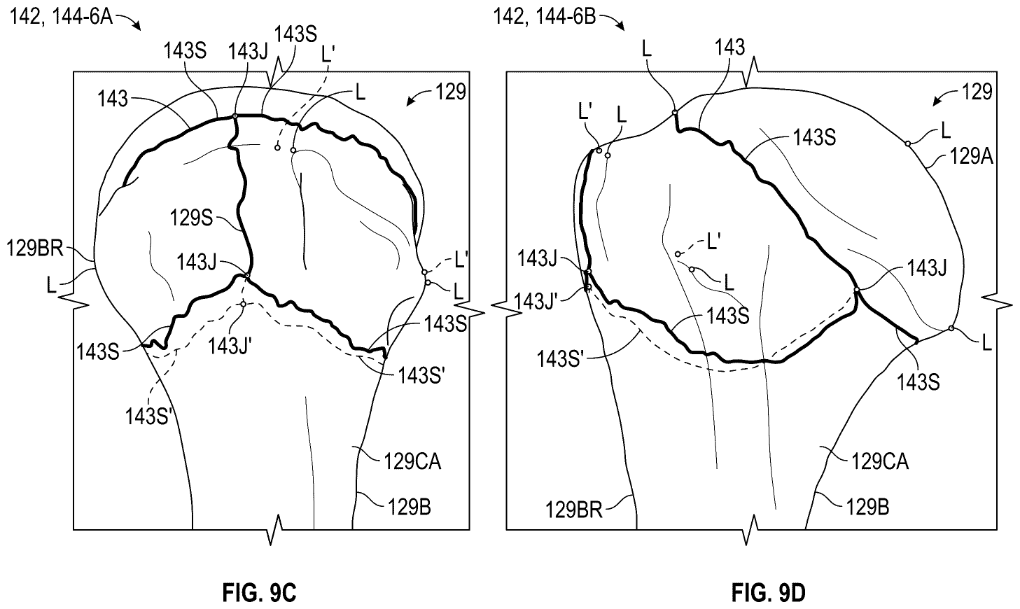

Referring to FIGS. 9A-9B, with continuing reference to FIGS. 2 and 8, aspects of the anatomical model 129 of FIG. 8 are shown. Each fracture pattern 143 may be generated automatically and/or in response to user interaction with the user interface 142. In implementations, the user may interact with the user interface 142 to manually specify a geometry of the fracture pattern 143.

Each fracture pattern 143 may include one or more segments 143S. The fracture pattern 143 may include two or more segments 143S that may be continuous or may be spaced apart from each other. Two or more of the segments 143S may meet at one or more junctions 143J. Each segment 143S may be a continuous loop and/or may be established between a pair of junctions 143J. In implementations, each of the segments 143S may extend along a surface of a portion of the bone volume 129B such as an external surface of the cancellous bone volume 129CA. In other implementations, one or more of the segments 143S may extend along a surface of the cortical bone volume 129CO (see, e.g., FIG. 7). Each of the segments 143S may be a linear or non-linear path extending between two junctions 143J. In implementations, each of the segments 143S may include one or more undulations. The undulations may be representative of a fracture line observed in prior case(s) and/or hypothetical case(s) based on empirical data, parametric modeling, etc.

Referring to FIGS. 9C-9D, with continuing reference to FIGS. 2, 8 and 9A-9B, various techniques may be utilized to establish the fracture pattern 143. The spatial module 137 may be adapted to establish or identify one or more landmarks L relative to the bone volume 129B and/or another portion of the virtual anatomical model 129. In implementations, the comparison module 138 may be adapted to determine one or more landmarks L based on a comparison of the anatomical model 129 and one or more prior cases. In implementations, the surgeon or clinical user may interact with the display window 144, such as the display window 144-6A or 144-6B to adjust a position of one or more landmarks L (see, e.g., landmarks L′). The spatial module 137 may be adapted to adjust the position of one or more segments 143S in response to adjusting one or more associated landmarks L (see, e.g., segments 143S′ and association junctions 143J′).

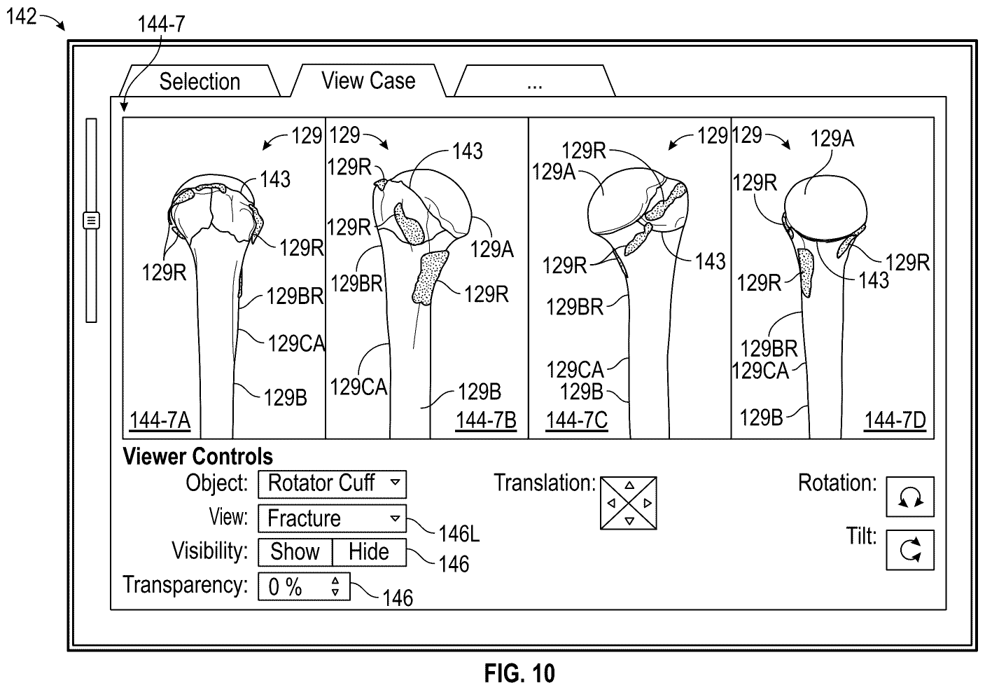

The display model 136 may be adapted to display an isolated view of the fracture pattern 143 relative to the bone volume 129B and/or attachment regions 129R. In the implementation of FIG. 10, the surgeon or clinical user may interact with one or more display windows 144-7 such as a seventh set of display windows 144-7A to 144-7D, or another portion of the user interface 142 such as one of the lists 146L, to hide the representation of the cortical bone volume 129CO (see FIG. 7). Selectively hiding the cortical bone volume 129CO may assist the surgeon or clinical user in observing a relative position between the fracture pattern 143 and attachment region(s) 129R.

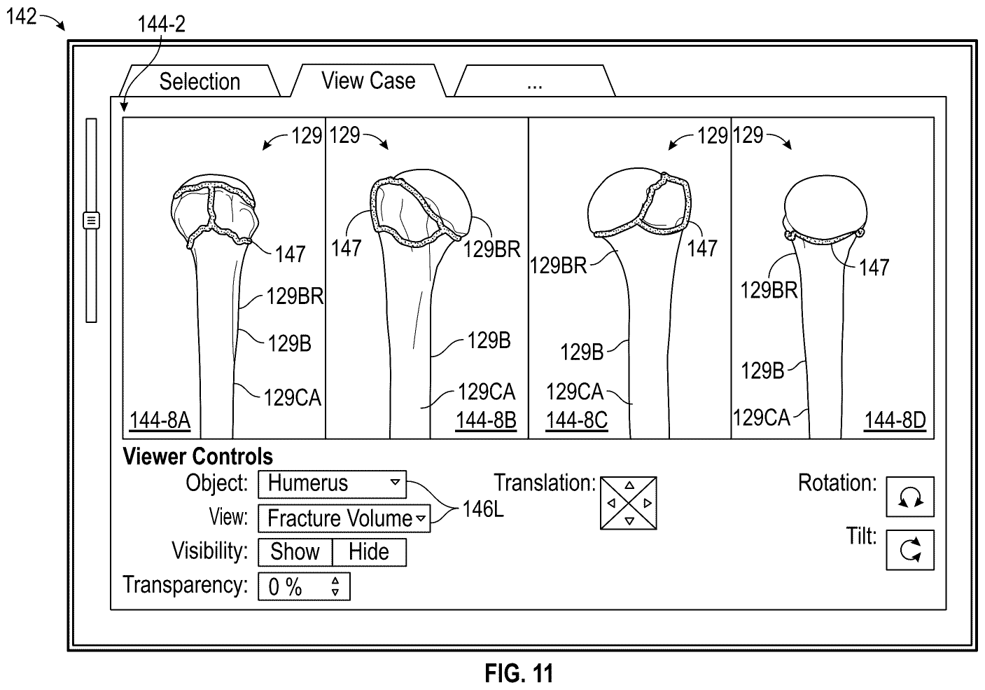

Referring to FIG. 11, with continuing reference to FIGS. 2 and 9A-9D, various techniques may be utilized to establish the fracture pattern 143 relative to the virtual anatomical model 129. The display module 136 may be configured to display the virtual anatomical model 129 in one or more display windows 144-8, such as an eighth set of display windows 144-8A to 144-8D.

The spatial module 137 may be configured to generate a virtual fracture volume 147, which may be associated with a fracture pattern 143. The virtual fracture volume 147 may substantially or generally follow a length of a respective fracture pattern 143 (see, e.g., FIG. 10). For the purposes of this disclosure, the term “substantially” means±10 percent of the stated relationship or value unless otherwise indicated. A configuration (e.g., definition) 145 (FIG. 2) may be established according to the virtual fracture volume 147.

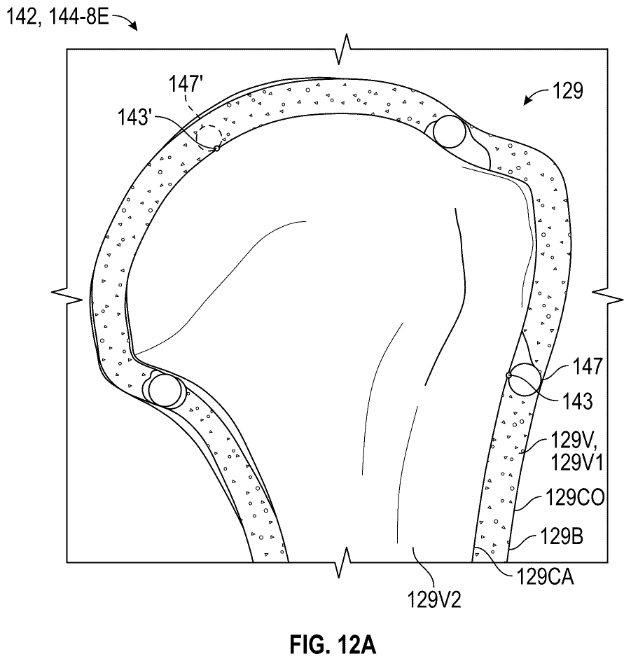

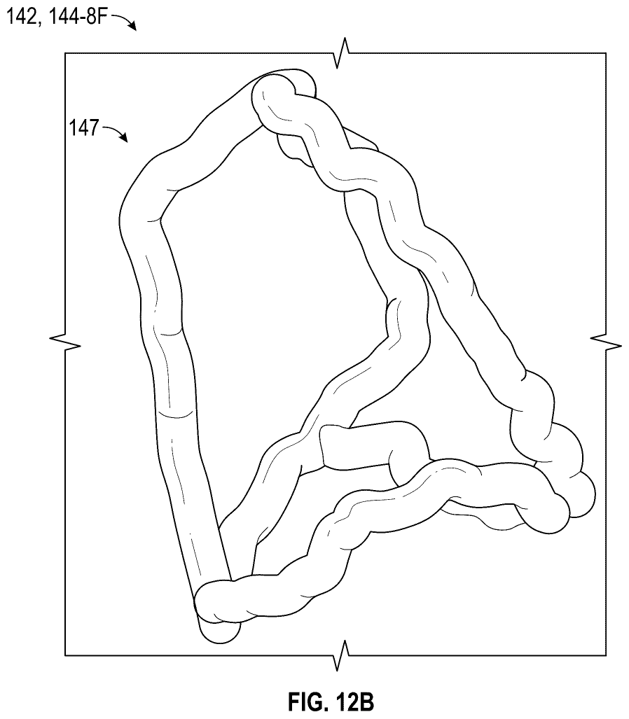

Referring to FIG. 12A, with continuing reference to FIGS. 2 and 11, various techniques may be utilized to establish the virtual fracture volume 147. The display module 136 may be adapted to display the virtual fracture volume 147 in one or more display windows 144, such as display windows 144-8E, 144-8F (FIG. 12B). The virtual fracture volume 147 may be established by extruding a shape along a length of the fracture pattern 143. The display module 136 may be adapted to display the virtual fracture volume 147 in the display windows 144-8E, 144-8F. Various shapes may be utilized, such as a straight or curved line segment, an ellipse (e.g., circle), a polygon (e.g., rectangle) and/or complex shape. A geometry of the virtual fracture volume 147 may be selected to facilitate severing of a physical anatomical model. In implementations, the virtual fracture volume 147 may be dimensioned to span between an external surface of the cancellous bone volume 129CA and an external surface of the cortical bone volume 129CO. FIG. 12B discloses an isolated view of the virtual fracture volume 147 of FIG. 12A in the display window 144-8F. In other implementations, fracture volume 147′ may be spaced apart from the external surface of the cortical bone volume 129CO (volume 147′ shown in dashed lines in FIG. 12A).

The fracture volume 147 may have various constructions. The fracture volume 147 may be homogenous or may have two or more heterogenous regions. In implementations, the fracture volume 147 may be substantially hollow or may include one or more voids that may serve to weaken a localized region of an associated physical anatomical model.

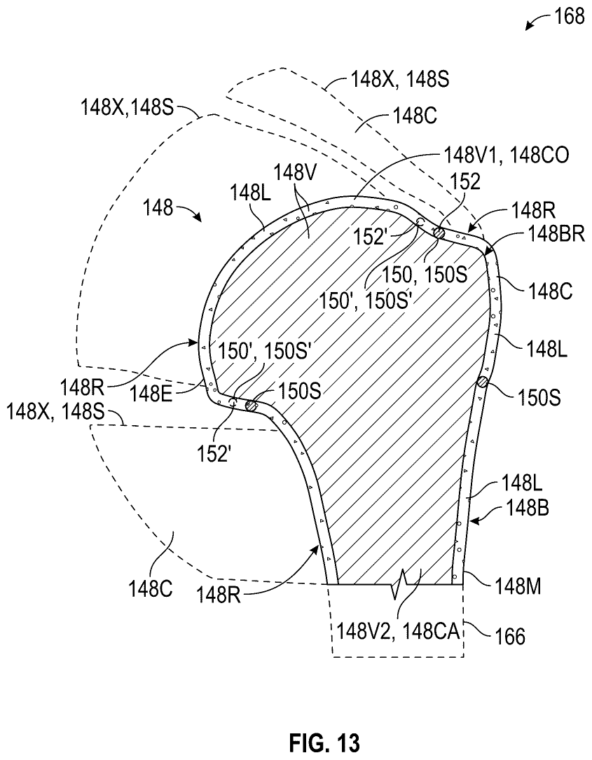

Referring to FIG. 13, with continuing reference to FIGS. 2 and 12A-12B, the virtual anatomical model 129 may be utilized to establish a physical anatomical model 148. The comparison module 138 may be configured to generate one or more configurations 145 (FIG. 2) associated with the virtual anatomical model(s) 129. The comparison module 138 may be adapted to generate the configuration 145 in response to specifying one or more parameters associated with a respective virtual anatomical model 129, including any of the parameters disclosed herein such as parameter(s) of the fracture classification scheme 141.

The configuration 145 may specify various information for forming an instance of an associated physical anatomical model 148, which may be based on a respective virtual anatomical model 129. The configuration 145 may include one or more files in a predetermined data structure or format. In implementations, the configuration 145 may include a coordinate set and/or other information such as material selection(s) associated with volume(s) of the physical anatomical model 148. Each physical anatomical model 148 may be formed utilizing various techniques, including any of the techniques disclosed herein such as rapid prototyping (e.g., printing) and other additive manufacturing techniques, casting, machining, etc.

The configuration 145 may specify a fracture path (e.g., fracture pattern) 150 that may be associated with a physical anatomical model 148. Each fracture path 150 may be established according to an assigned fracture pattern 143 such that the fracture patterns 143 may be reproduceable. The configuration 145 may specify coordinate data and/or other information to establish the fracture path 150 according to the assigned fracture pattern 143. The configuration 145 may be generated such that the respective physical anatomical model 148 may be severable along the fracture path 150 to establish one or more fragments to establish a fragmentary state of the physical anatomical model 148.

The physical anatomical model 148 may include a main body 148M. For the purposes of this disclosure, the alphanumeric suffixes associated with each indicator of the virtual anatomical models are utilized in a like manner in describing similar aspects of the physical anatomical models unless otherwise indicated. The main body 148M may include an external surface 148E associated with an anatomical profile of a bone, including any of the bones disclosed herein. In implementations, the anatomical profile of the bone may be associated with a long bone, such as a humerus, femur, fibula, or tibia. The physical anatomical model 148 may be secured to at least one fixture 166 to establish an assembly 168 (shown in dashed lines).

The physical anatomical model 148 may include one or more physical components 148C. Each component 148C may be representative of an associated component 129C of the respective virtual anatomical model 129. The components 148C of the physical anatomical model 148 may include any of the components 129C of the respective virtual anatomical model 129, such as bone volume 148B. A representation of one or more of the components 129C may be omitted from the physical anatomical model 148 to provide tailored training to the surgeon or clinical user (e.g., different difficulty level, etc.).

The physical anatomical model 148 may include one or more extensions 148X (shown in dashed lines). Each extension 148X may extend from the external surface 148E of the main body 148M. One or more of the extensions 148X may be representative of respective soft tissue volume(s) 148S, including any of the soft tissue disclosed herein. The soft tissue volume 148S may be attached to the bone volume 129B at a respective attachment region 148R.

The main body 148M of the physical anatomical model 148 may include one or more volumes 148V. The main body 148M may include a first volume 148V1 and a second volume 148V2. The first volume 148V1 may establish the external surface 148E of the main body 148M. The first volume 148V1 may be representative of cortical bone. The second volume 148V2 may be representative of cancellous bone.

At least one fracture path 150 may be established along the physical anatomical model 148. The fracture path 150 may be established according to a predetermined fracture pattern 143 (see, e.g., FIG. 12A). The main body 148M may include the fracture path 150. The fracture path 150 may establish one or more localized regions 148L of the physical anatomical model 148. The fracture path 150 may divide the main body 148M into one or more localized regions 148L. The fracture path 150 may include one or more segments 150S. Each of the segments 150S may establish a loop about a respective localized region 148L (see also fracture volume 152). The external surface 148E along at least one of the localized regions 148L may be associated with an articular surface of a joint, including any of the joints and bones disclosed herein. Each extension 148X may extend from the external surface 148E of the main body 148M adjacent to one or more segments 150S of the fracture path 150.

The main body 148M of the physical anatomical model 148 may include at least one, or more than one, physical fracture volume 152. The physical fracture volume 152 may be established along the fracture path 150. The main body 148M may be severable along the fracture volume 152 to establish one or more fragments. The physical fracture volume 152 may establish frangible connection(s) between the localized regions 148L and each other and/or the main body 148M of the physical anatomical model 148.

The fracture path 150 may extend along a boundary region 148BR between adjacent volumes 148V of the physical anatomical model 148, such as between the first volume 148V1 and second volume 148V2. The fracture volume 152 may be established along the fracture path 150 such that the fracture volume 152 may be at least partially embedded in one or more of the volumes 148V, such as the first volume 148V1 of the main body 148M. The main body 148M may be severable along the fracture volume 152 to establish the one or more fragments. In implementations, the physical fracture volume 152 may be spaced apart from the external surface of the main body 148M of the physical anatomical model 148 (see, e.g., fracture volume 147′ of FIG. 12A).

The volumes 148V of the physical anatomical model 148 may have various properties. The first volume 148V1 may have a first property. The second volume 148V2 may have second property. The fracture volume 152 may have a third property. The first, second and/or third properties may be the same or may differ from each other. The first, second and third properties may include respective first, second and third material strengths. The second and/or third material strengths of the second volume 148V2 and fracture volume 152 may be less than the first material strength of the first volume 148V1. The first material strength may be representative of cortical bone. The second material strength may be representative of cancellous bone. The lesser material strength may establish relatively weaker region(s) in the physical anatomical model 148 to promote fragmentation of the physical anatomical model 148 in a reproduceable manner. The fracture volume 152 may incorporate any of the materials disclosed herein, such as a silica-based material.

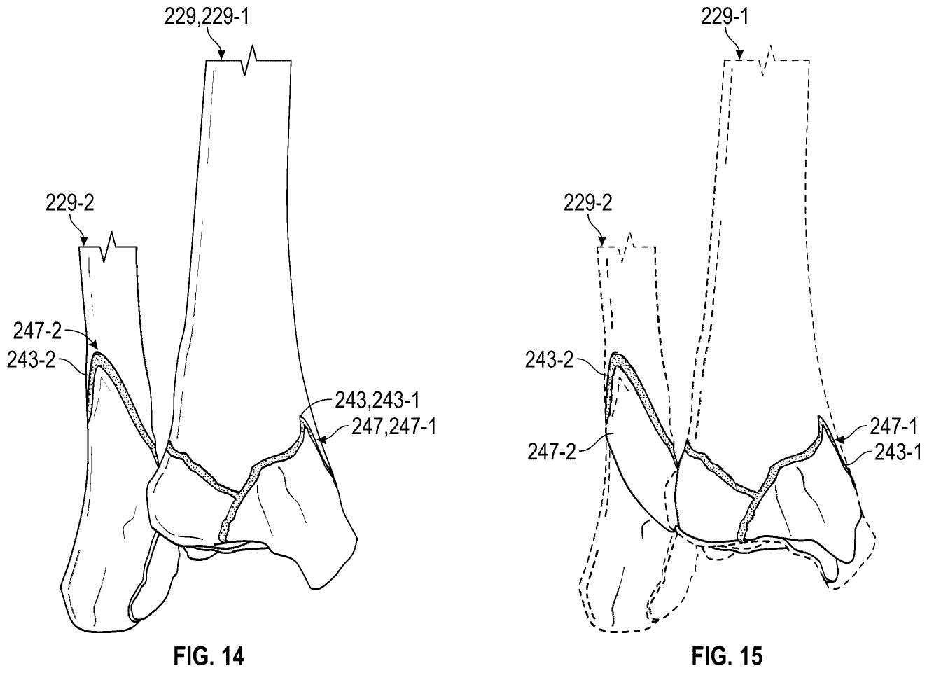

FIGS. 14-15 disclose virtual anatomical models 229 according to another implementation. The anatomical models 229 may include a first anatomical model 229-1 and a second anatomical model 229-2, which may be adjacent to the first anatomical model 229-1. FIG. 15 depicts the anatomical models 229 in phantom. The anatomical models 229 may incorporate any of the features disclosed herein, including anatomical features representative of anatomy, such as one or more bones including cartilage, cortical and/or cancellous bone tissue, soft tissue including muscle, ligaments and/or tendons, etc., and/or other tissue. Each anatomical model 229 may be associated with a surgical plan (e.g., surgical plan 131 of FIG. 2). In implementations, the anatomical models 229 may be associated with respective bones of a limb of a patient. The bones may be adjacent to each other. The anatomical model 229-1 may include portions associated with a joint of a patient, including any of the joints disclosed herein such as an ankle joint. The anatomical model 229-1 may be associated with a tibia of a patient. The anatomical model 229-2 may be associated with a fibula of the patient.

The anatomical models 229-1, 229-2 may be associated with respective virtual fracture volumes 247 (indicated by 247-1, 247-2). Each fracture volume 247-1, 247-2 may be established by a respective fracture pattern 243 (indicated by 243-1, 243-2). The fracture pattern 243 may be established utilizing any of the techniques disclosed herein. The fracture pattern 243 and associated fracture volume 247 may be established based on an associated fracture classification scheme 141 (FIG. 2). In implementations, the fracture pattern 243 may be dimensioned to substantially follow an outer periphery of the respective anatomical model 229. The outer periphery may be associated with an inner cortical wall or an outer cortical wall of a bone. The fracture volume 247 may be dimensioned to span between opposite sides of a perimeter of the fracture pattern 243 such that the fracture volume 247 may extend substantially through a main body of the virtual anatomical model 229.

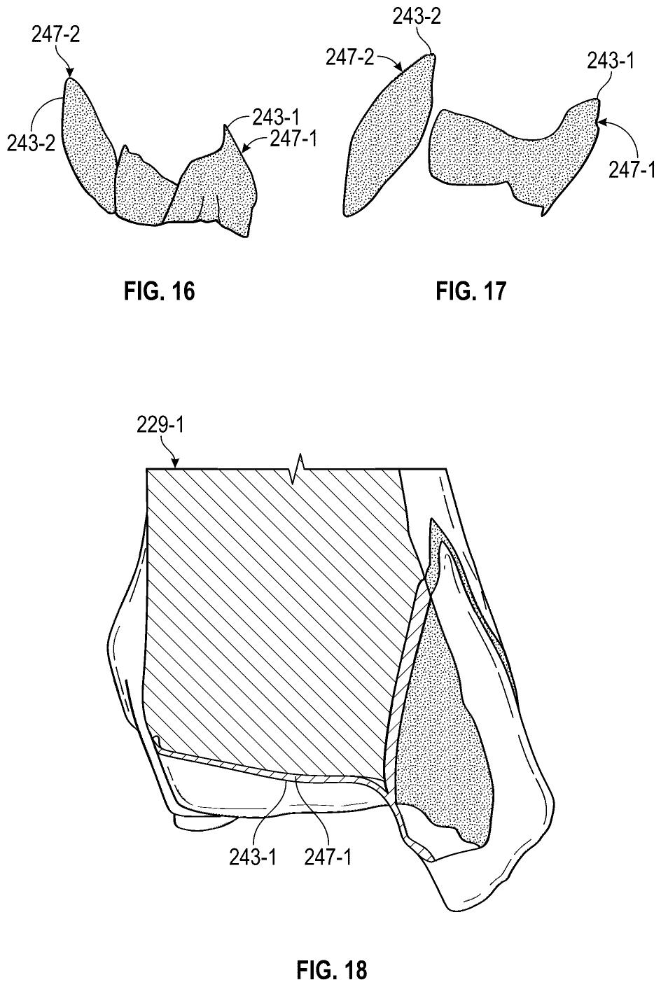

A portion of the virtual fracture volume 247 may be established by extruding a shape along a length of the perimeter of the fracture pattern 243. The fracture volume 247 may include a portion (e.g., area) within a periphery of the fracture pattern 243 such that the fracture volume 247 may have a contiguous (e.g., enclosed) three-dimensional profile. The fracture volume 247 may have various geometries, such as a substantially planar or complex geometry. In the implementation of FIG. 15, each of the fracture volumes 247 has a contoured geometry associated with a profile of the fracture pattern 243. FIG. 16 discloses the fracture volumes 247-1, 247-2 at the orientation of FIGS. 14-15. FIG. 17 discloses the fracture volumes 247-1, 247-2 at a different orientation.

Various techniques may be utilized to establish a geometry of the portion of the fracture volume 247 inside of the perimeter of the fracture pattern 243, such as manual sculpting or automated techniques. Automated techniques may include a “close holes” operation in which an interior of an object is filled by a two-dimensional or three-dimensional mesh. The spatial module 137 (FIG. 2) may be configured to generate the fracture pattern 243 and associated fracture volume 247 utilizing any of the techniques disclosed herein.

FIG. 18 discloses a sectional view of the virtual anatomical model 229-1 and the respective fracture volume 247-1. In the implementation of FIG. 18, the fracture volume 247-1 may extend completely, or at least substantially, through a volume of the anatomical model 229-1. The fracture volume 247-1 may be established in the anatomical model 229-1 to facilitate at least partial or complete separation of adjacent portions of an associated physical anatomical model. The virtual anatomical model 229 may be utilized to establish a physical anatomical model utilizing any of the techniques disclosed herein. The anatomical model 229 and fracture volume 247 may be associated with the same and/or different structures, materials, porosities, etc., including any of those disclosed herein, for establishing the physical anatomical model. A configuration 145 (FIG. 2) for fabrication the physical anatomical model may specify a geometry associated with the fracture volumes 247-1, 247-2 for establishing a physical fracture path (e.g., pattern) and associated fracture volume in the physical anatomical model.

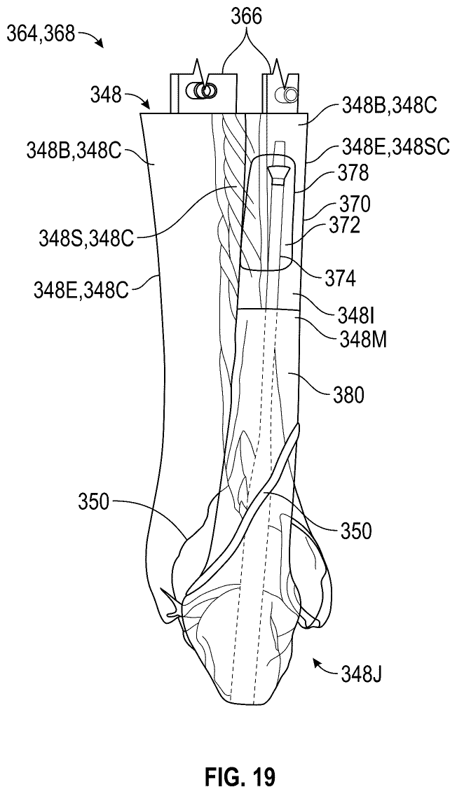

FIG. 19 discloses a system 364 for an orthopaedic procedure. The system 364 may incorporate any of the features disclosed herein. The system 364 may include a physical anatomical model 348. The physical anatomical model 348 may include one or more physical components 348C. The physical anatomical model 348 may be secured to at least one fixture 366 to establish an assembly 368. A main body 348M of the physical anatomical model 348 may be representative of anatomy. In implementations, the main body 348M may be associated with an anatomical profile of one or more bones, including any of the bones disclosed herein. In implementations, the bone may be a long bone, such as a fibula, tibia, femur or humerus.

The main body 348M of the physical anatomical model 348 may include one or more physical bone volumes (e.g., components) 348B. The bone component 348B may include an exterior 348E having a surface contour 348SC representative of the respective bone. The main body 348M of the physical anatomical model 348 one or more soft tissue components 348S on the bone component(s) 348B. The soft tissue component 348S may be representative of soft tissue.

The bone component 348B may include a first portion 370 and/or a second portion 380. The first portion 370 may be adjacent to the second portion 380. In implementations, the first portion 370 may be incorporated into the second portion 380, or vice versa. The first portion 370 and second portion 380 may be a unitary structure or may be separate and distinct components that may be attached or otherwise secured to each other. The first portion 370 and/or second portion 380 may include a joint region 348J of the bone component 348B. The joint region 348J may be representative of an orthopaedic joint, including any of the joints disclosed herein, such as an ankle joint. The first portion 370 and/or second portion 380 may establish the exterior 348E of the bone component 348B. The first portion 370 may be adjacent to an intermediate portion and/or a proximal portion of the bone component 348B, and the second portion 380 may be adjacent to a distal portion of the bone component 348B, or vice versa. The first portion 370 may be distal to the second portion 380, or vice versa. In implementations, the bone component 348B of the physical anatomical model 348 may be representative of a fibula. The second portion 380 may be associated with a distal portion of the representative fibula. The first portion 270 may be associated with an intermediate and/or proximal portion of the representative fibula.

The physical anatomical model 348 may incorporate one or more features for viewing an interior 3481 of the model 348. The physical anatomical model 348 may incorporate various materials to facilitate viewing the interior 3481. In implementations, the first portion 370 may include a first material. The second portion may include a second material. The first and second materials may be the same or may differ with respect to one or more material characteristics. The first material may be translucent or may be completely or substantially transparent. The first portion 370 may be formed of the first material such that an interior portion 372 of the body 348M, including the interior 3481 of the bone component 348B, may be viewed through the first material from outside of the physical anatomical model 348. The second material associated with the second portion 380 may have a transparency that may be less than a transparency of the first material associated with the first portion 370. The second material associated with the second portion 380 may have an opacity that may be greater than an opacity of the first material associated with the first portion 370. The second material may be substantially or completely opaque. For the purposes of this disclosure, the term “transparent” refers to the physical property of allowing light to pass through the material without appreciable scattering of light. The term “translucent” is used herein to describe elements that are both partially reflective and partially transmitting of light incident on them. An “opaque” material is a material that does not allow light to pass through, blocking the transmission of light.

The physical anatomical model 348 may include at least one a viewing portal (e.g., window) 378 for viewing the interior 3481 of the bone component 348B from outside the physical anatomical model 348. The first portion 370 and/or another portion of the main body 348M may include the viewing portal 378. In implementations, the viewing portal 378 may be relatively more transparent or translucent than one or more other portions of the main body 348M of the physical anatomical model 348. In implementations, the first material of the first portion 370 may establish the viewing portal 378. A perimeter of the viewing portal 378 may be bounded by a material that may be opaque or may otherwise have a relatively lesser transparency than the first material establishing the viewing portal 378. In implementations, the second portion 380 may substantially surround the perimeter of the viewing portal 378.

The interior portion 372 of the main body 348M viewable through the viewing portal 378 and/or another section of the first portion 370 may be an interior area of interest associated with an orthopaedic procedure. In implementations, the interior portion 372 of the bone component 348B may include an internal cavity 374. The cavity 374 may be at least partially bound by the transparent or translucent material. Some, or all, of the cavity 374 may be viewable through the first portion 370 from outside of the model 348. The cavity 374 may include a portion in the shape of a longitudinal (e.g., intramedullary) canal.

The internal cavity 374 may be dimensioned to at least partially receive an orthopaedic implant (e.g., orthopaedic implant 382 of FIG. 21). The cavity 374 may extend at least partially through the first portion 370 and/or the second portion 380 of the bone component 348. In implementations, the cavity 374 may extend inwardly from the exterior 348E of the bone component 348B, such as the joint region 348J. The internal cavity 374 may be at least partially aligned with the viewing portal 378.

The internal cavity 374 may have various geometries. In implementations, the cavity 374 may be representative of a reaming path for receiving an intramedullary nail. The cavity 374 may be formed with the physical anatomical model 348 utilizing any of the techniques disclosed herein, such as through rapid prototyping (e.g., printing) and other additive manufacturing techniques. In other implementations, the cavity 374 may be established subsequent to forming the physical anatomical model 348. The cavity 374 may be established using a machining or other material removal technique. The surgeon or other user may utilize a surgical instrument such as a drill to establish the cavity 374.

The bone component(s) 348B and/or another portion of the physical anatomical model 248 may include one or more fracture paths 350. The fracture paths 350 may establish one or more localized regions. The bone component 348B may be severable along the fracture path 350 to establish one or more fragments associated with a respective one of the localized regions. The fracture paths 350 may be established utilizing any of the techniques disclosed herein. The cavity 374 may extend through one or more of the fracture paths 350 (e.g., FIG. 19). The cavity 374 may be at least partially aligned with the viewing portal 378, which may be spaced apart from the facture path 350.

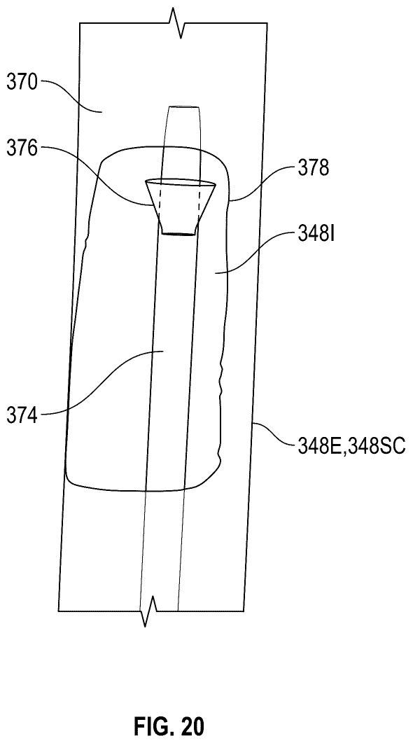

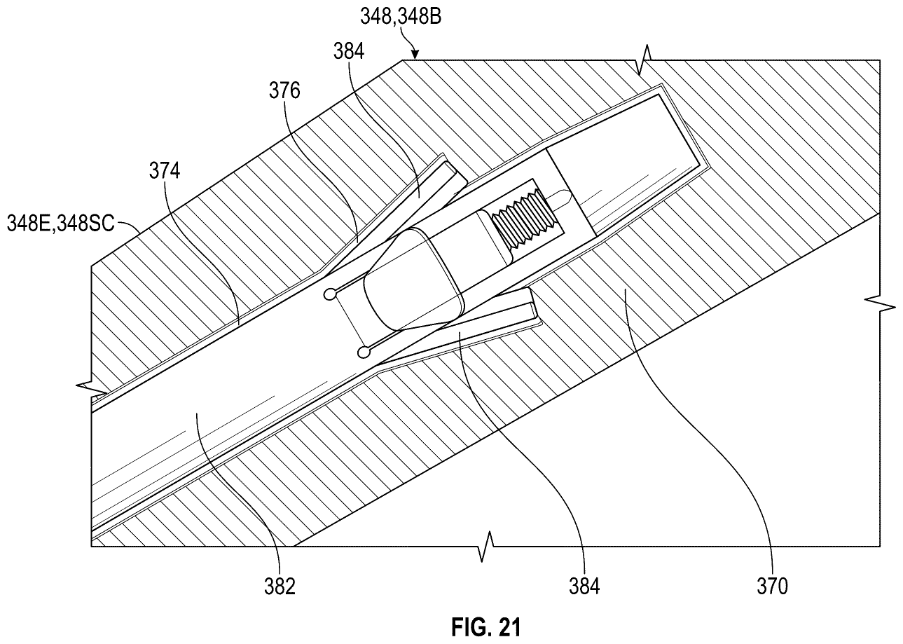

Referring to FIG. 20, with continuing reference to FIG. 19, the cavity 274 may include a deployment region 376. The deployment region 376 may be dimensioned to at least partially receive a (e.g., deployable) portion of an orthopaedic implant movable between an undeployed state and a deployed state (e.g., implant 382 of FIG. 21). The deployment region 376 may be at least partially aligned with the viewing portal 378 such that the deployment region 376 may be viewable through the viewing portal 378.

The deployment region 376 may be an (e.g., localized) area of interest associated with an orthopaedic procedure. The deployment region 376 may be established in or otherwise adjacent to the first portion 370 such that the deployment region 376 may be viewed from outside of the physical anatomical model 348. In implementations, the deployment region 376 may include a conical void. The conical void may be associated with a geometry of the portion of the implant in the deployed state (e.g., FIG. 21).

Referring to FIG. 21, with continuing reference to FIGS. 19-20, an orthopaedic implant 382 may be at least partially receivable within the internal cavity 374. The implant 382 may include one or more components that may be situated at a surgical site, including plates, anchors, screws, nails, suture, grafts, etc. In implementations, the implant 382 may be an intramedullary (e.g., fibular) nail. The implant 382 may include a portion having an undeployed state and a deployed state. In implementations, the portion of the implant 382 may include one or more deployable (e.g., bendable) members (e.g., anchors or talons) 384. The deployable members 384 may be movable between the undeployed state (e.g., position) and the undeployed state (e.g., position). The deployment region 376 may be dimensioned to receive the portion of the implant 382 including the deployable members 384. The deployable members 384 may be adapted to deploy outward from an undeployed position to a deployed position within the deployment region 376 of the cavity 374 to secure the implant 382 to the bone component 348B. In the implementation of FIGS. 19-20, deployment of the deployable members 384 of the implant 382 to the deployed position may be viewable through the first portion 370 of the bone component 348B. Being able to view the deployment of the implant may assist a surgeon in training, rehearsing and/or evaluating an orthopaedic procedure.

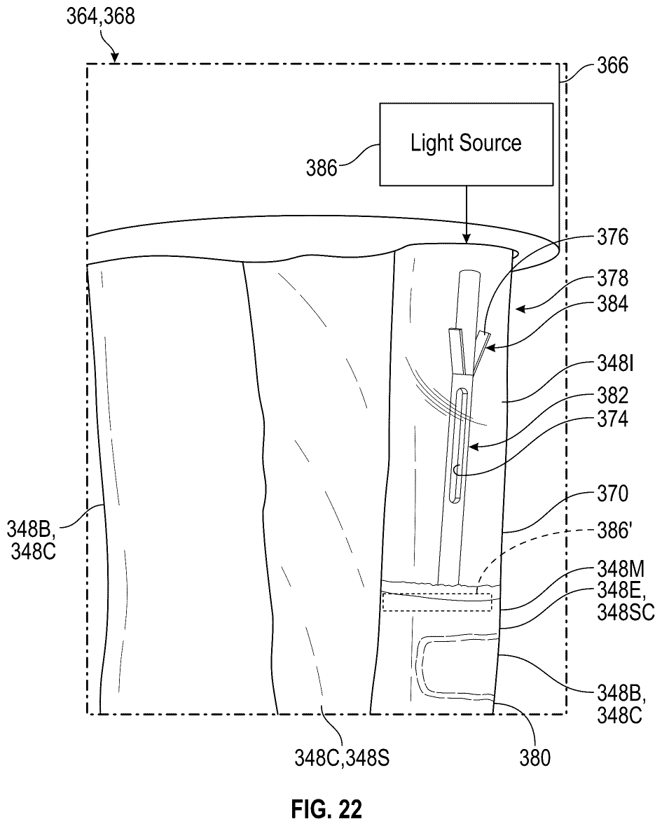

Referring to FIG. 22, with continuing reference to FIGS. 19-21, the system 364 may include one or more light sources 386. The light source(s) 386 may be operable to illuminate the interior (e.g., interior portion) 3481 of the physical anatomical model 348, including at least a portion of the first material of the first portion 370, the internal cavity 374 and/or the orthopaedic implant 382. The interior 3481 may be viewable through the first material. The light source(s) 386 may be positioned at various locations relative to the physical anatomical model 348. The light source 386 may be at least partially embedded in the main body 348M of the physical anatomical model 348, including the first portion 370 and/or another portion of the bone component 348B (e.g., light source 386′ shown in dashed lines in FIG. 22). In other implementations, the light source 386 may be external to the main body 348M. The light source 386 may be operable to illuminate the first portion 370 of the bone component 348B adjacent to the viewing portal 378. Illumination may allow for relatively clearer viewing, which may assist the surgeon or user in positioning, deploying and/or evaluating the implant 382 in the cavity 374.

Various light sources may be utilized to illuminate the physical anatomical models and systems disclosed herein, including incandescent, fluorescent, halogen, and/or light emitting diodes (LED). The light source 386 may be coupled to a power supply. The power supply may be external or may be embedded in the physical anatomical model (e.g., battery powered).

The light source 386 may be configured to generate light in one or more frequencies and/or frequency ranges of visible and/or non-visible light. The frequencies and/or frequency ranges may be defined in a visual light spectrum (e.g., 400 nm to 700 nm), near infrared light spectrum (e.g., 2.5 μm to 750 nm) and/or infrared light spectrum (e.g., 25 μm to 2.5 μm). The light source 256 may be configured to generate light characterized by various hue, saturation and/or brightness. The light source 386 may include a plurality of modes associated with distinct frequencies and/or frequency ranges of visible and/or non-visible light. The plurality of modes may include first and second modes associated with first and second frequency ranges, respectively.

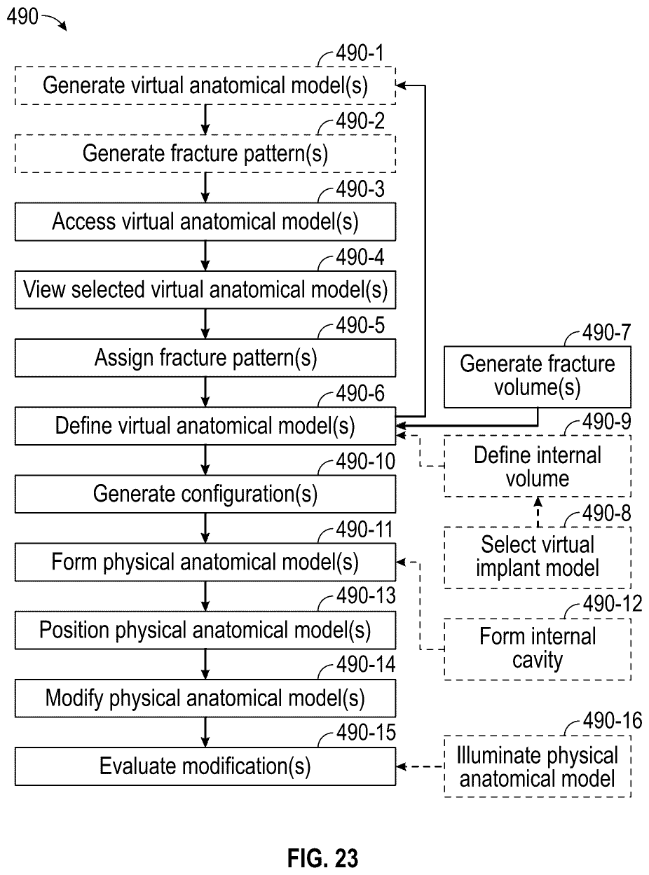

FIG. 23 discloses a method in a flowchart 490 for a surgical procedure. The method 490 may be utilized to pre-operatively plan, rehearse and/or train for various orthopaedic procedures, such as an arthroplasty for restoring functionality to shoulders, ankles, knees, hips and other joints. The method 490 may be utilized with any of the planning systems, virtual anatomical models and/or physical anatomical models disclosed herein. The method 490 may be utilized to establish physical anatomical model(s) for training and rehearsing for a surgical procedure. The method 490 may be utilized to evaluate the accuracy in which a surgeon may implement a surgical procedure on a physical anatomical model associated with an anatomy of a patient or hypothetical case. Fewer or additional steps than are recited below could be performed within the scope of this disclosure, and the recited order of steps is not intended to limit this disclosure. Reference is made to the system 120 and user interface 142 for illustrative purposes.

Referring to FIG. 2, with continuing reference to FIG. 23, at step 490-1 one or more virtual anatomical models 129 may be generated. Each virtual anatomical model 129 may be associated with an anatomy of a patient and/or hypothetical case. The virtual anatomical models 129 may be generated utilizing any of the techniques disclosed herein. The virtual anatomical models 129 may include any of the anatomies and tissue types disclosed herein, including bone, ligament, tendon, cartilage, etc. At step 490-2, one or more fracture patterns 143 may be generated. The fracture patterns 143 may be generated utilizing any of the techniques disclosed herein.

At step 490-3, one or more virtual anatomical models 129 may be accessed. The virtual anatomical model 129 may be accessed from memory 134, the network 23 and/or another computing device, such as the storage system 27 (FIG. 1). Step 490-3 may include selecting one or more virtual anatomical models 129 from a set of virtual anatomical models 129. Each virtual anatomical model 129 may be associated with an anatomy. Various techniques may be utilized to select the virtual anatomical model 129. The virtual anatomical models 129 may be stored in memory of a computing device, such as in the database 128 or the memory 134 of the computing device 132.

Referring to FIG. 3, with continuing reference to FIGS. 2 and 23, selecting the virtual anatomical model 129 may include selecting or otherwise specifying various parameters associated with the set of virtual anatomical models 129. The parameters may include any of the parameters disclosed herein, including anatomy, patient classification, fracture classification and/or case. The parameters may be selected in response to user interaction with the graphical user interface 142.

Referring to FIGS. 4-7, with continuing reference to FIGS. 2 and 23, at step 490-4 the selected virtual anatomical model(s) 129 may be viewed in the graphical user interface 142. Step 490-4 may include setting the parameter(s) in response to user interaction with the graphical user interface 142. The parameters may be specified in response to the surgeon or clinical user interacting with the user interface 142. One or more of the parameters may be associated with predefined fracture classification scheme(s) 141. The fracture classification schemes 141 may include any of the fracture classification schemes disclosed herein.

Referring to FIGS. 9A-9D, with continuing reference to FIGS. 2-3, 8 and 23, one or more fracture patterns 143 may be assigned to the virtual anatomical model 129 at step 490-5. The fracture pattern 143 may be assigned to the virtual anatomical model 129 utilizing any of the techniques disclosed herein. The fracture pattern 143 may be assigned to the virtual anatomical model 129 based on one or more parameters, including any of the parameters disclosed herein such as anatomy, patient classification, fracture classification and/or case. Assigning the fracture pattern 143 may occur in response to setting one or more parameters associated with the fracture classification scheme 141. Step 490-5 may include causing the virtual anatomical model 129 and/or the assigned fracture pattern 143 to be displayed in one or more display windows 144 of the graphical user interface 142, such as the display windows 144-6A to 144-6D (FIG. 8).

At step 490-6, aspects of one or more of the virtual anatomical models 129 may be defined. Each virtual anatomical model 129 may be defined prior, during and/or subsequent to generating the virtual anatomical model(s) 129 at step 490-1, generating the fracture pattern(s) 143 at step 490-2, selecting the virtual anatomical model(s) 129 at step 490-3, viewing the selected virtual anatomical model(s) 129 at step 490-4 and/or assigning fracture pattern(s) 143 to the virtual anatomical model(s) 129 at step 490-5. Defining the virtual anatomical model 129 may include setting one or more parameters of the virtual anatomical model 129, including any of the parameters disclosed herein. The parameters may be selected in response to user interaction with the graphical user interface 142 (e.g., FIG. 3). The parameters may be associated with one or more fracture classifications 141 (FIG. 2) and/or fracture patterns 143 (e.g., FIGS. 2 and 9).

Referring to FIGS. 11 and 12A-12B, with continuing reference to FIGS. 2 and 23, defining the virtual anatomical model 129 may include generating one or more virtual fracture volumes 147 at step 490-7. Each virtual fracture volume 147 may be established utilizing any of the techniques disclosed herein.

Defining the virtual anatomical model 129 at step 490-6 may include selecting at least one virtual implant model 30 (FIG. 2) at step 490-8. The implant model 30 may be associated with a physical orthopaedic implant suitable for treating the anatomy associated with the virtual anatomical model 129. The implant model 30 may be associated any of the implants disclosed herein, such as the implant 382 (FIG. 21). At step 490-9, an internal volume may be defined in the virtual anatomical model 129. The internal volume may be dimensioned to accommodate a profile of the implant model 30 based on a position of the implant model 30 in the virtual anatomical model 129.

At step 490-10, one or more configurations (e.g., definitions) 145 may be generated. Each configuration 145 may be associated with at least one virtual anatomical model 129, fracture pattern 143, physical anatomical model 148, fracture path 150 and/or physical fracture volume 152. Each configuration 145 may be generated utilizing any of the techniques disclosed herein. The configuration 145 may be associated with a physical anatomical model 148 that may be representative of a selected virtual anatomical model 129. Each configuration 145 may be generated in response to selecting the respective virtual anatomical model 129 at step 490-3, assigning the respective fracture pattern 143 at step 490-5 and/or defining the selected virtual anatomical model 129 at step 490-6. The configuration may be established according to the selection or setting of any parameters associated with the selected virtual anatomical model 129, including any fracture pattern 143 and/or fracture volume 152. The configuration 145 may include data and other information sufficient to establish a physical anatomical model 148 based on the parameters of the selected virtual anatomical model 129, including coordinate information, color, texture and/or moduli of elasticity of the associated tissues, geometry associated with one or more fracture paths 150, characteristics of the assigned materials including any of the material characteristics disclosed herein such as transparency or opacity, etc. The configuration 145 may specify one or more fracture paths 150 established in the physical anatomical model 148 according to the assigned fracture pattern 143. In implementations, the configuration 145 may include geometry associated with an internal cavity, such as the internal cavity 374 (e.g., FIGS. 19-21).

Referring to FIG. 13, with continuing reference to FIGS. 2, 12A-12B and 23, one or more physical anatomical models may be fabricated or otherwise formed at step 490-11, including any of the physical anatomical models disclosed herein, such as the physical anatomical model 148 (e.g., FIG. 13) and/or the physical anatomical model 348 (e.g., FIGS. 19-22). The physical anatomical model 148 may be formed to closely resemble or approximate a geometry of the associated anatomy, including bone and/or soft tissue. The physical anatomical model may be representative of any of the bones and joints disclosed herein, including a long bone such as a fibula, tibia, femur and/or humerus. In implementations, the surgeon may interact with the physical anatomical model 148 such that portion(s) of the physical anatomical model 148 may feel similar to soft (e.g., cancellous) bone tissue, muscle and other soft tissue, etc. The physical anatomical model 148 may be printed or otherwise formed according to the various parameters selected in the user interface 142. Various parameters may be utilized to form the physical anatomical model 148, including any of the parameters disclosed herein, such as density of bone and soft tissue, thickness of cortical bone, indicators, patient age, etc.

Each physical anatomical model 148 may be fabricated or otherwise formed based on a configuration 145 generated at step 490-11. Various materials may be utilized to form the physical anatomical models, including any of the materials disclosed herein. The physical anatomical model 148 including the main body 148M may incorporate metallic and/or non-metallic materials, including any of the materials disclosed herein such as a polymeric material. In implementations, the main body 148M may be formed from a substantially rigid material, such as a polymeric material, including photopolymers, silicones and thermoplastics. Portions of the physical anatomical model 148 may be formed from a relatively flexible material, including an elastomeric material such as rubber or silicone, to establish soft tissue volume(s) representative of any of the soft tissue disclosed herein. Portions of the physical anatomical model may include transparent and/or translucent material(s) for viewing an interior of the physical anatomical model, such as an interior (e.g., interior portion) 3481 of the physical anatomical model 348 (e.g., FIGS. 19-20 and 22).

Various techniques may be utilized to form the physical anatomical models. Each physical anatomical model may be formed utilizing any of the techniques disclosed herein, such as rapid prototyping (e.g., printing) and other additive manufacturing techniques, casting, machining, etc. The physical anatomical model may have a unitary construction or may have two or more components fixedly attached or otherwise secured to each other to establish a unit.

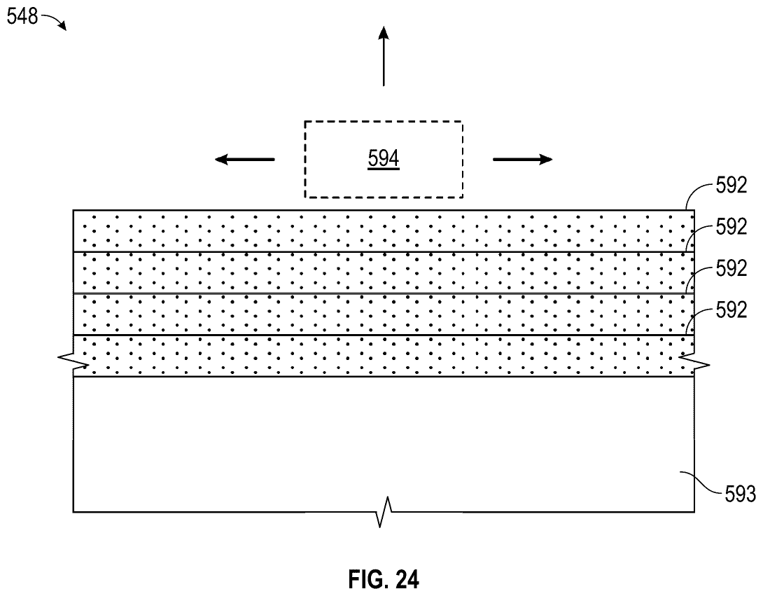

In the implementation of FIG. 24, one or more layers 592 of material may be printed or otherwise formed on a substrate 593 to establish a physical anatomical model 548. The physical anatomical model 548 may be representative of a virtual anatomical model, including any of the virtual anatomical models disclosed herein. A device 594 such as a three-dimensional printer may be configured to form the layers 592 according to data and other information associated with the respective configuration 145. The layers 592 of material may include any of the constructions, materials, color schemes, textures, porosities, etc. disclosed herein. The layers 592 may have respective moduli of elasticity that may substantially correspond to moduli of elasticity of respective biomaterial of the anatomy. The porosities of the material forming the physical anatomical model 548 may substantially approximate the porosity or density of the respective tissue. The physical anatomical model 548 may be formed with materials having different transparencies, translucence, opacity, etc., utilizing any of the techniques disclosed herein.

In the implementation of FIG. 13, the physical anatomical model 148 may include a first volume 148V1 and a second volume 148V2. The first volume 148V1 may be representative of cortical bone. The second volume 148V2 may be representative of cancellous bone. The fracture path 150 may establish one or more localized regions 148L of the physical anatomical model 148. The configuration 145 established at step 490-9 may specify a fracture volume 152 that may follow a length of the fracture path 150. The physical anatomical model 148 may be severable along the fracture path 150 and/or fracture volume 152 to establish one or more fragments associated with the respective localized regions 148L. The physical fracture volume 152 may establish a frangible connection between the localized region 148L and adjacent localized region(s) 148L and/or main body 148M of the physical anatomical model 148.

In the implementation of FIGS. 19-22, step 490-11 may include forming the physical anatomical model 348. The physical anatomical model 348 may include a body 348M associated with an anatomical profile of a bone. At least a first portion 370 of the body 348 may include a first material that may be transparent or translucent for viewing an interior (e.g., interior portion) 3481 of the body 348M. The body 348M may include an exterior 348E having a surface contour 348SC representative of a cortical wall of the bone. The body 348M may include a second portion 380 that may establish the exterior 348E adjacent to the first portion 370. The second portion 380 may include a second material having an opacity that may be greater than an opacity of the first material. The physical anatomical model 348 may be established such that the first portion 370 and/or another portion of the model 348 may include a viewing portal (e.g., window) 378 for viewing the interior 3481 from outside of the physical anatomical model 348.

Step 490-11 may include forming an internal cavity 374 in the interior 3481 of the physical anatomical model 348 at step 490-12. The internal cavity 374 may be dimensioned and formed according to any of the techniques disclosed herein. The first portion 370 may be at least partially aligned with the deployment region 376 of the internal cavity 374 such that the deployment region 376 may be visible from outside of the physical anatomical model 348. In implementations, the internal cavity 374 may extend through fracture path(s) 350 in the physical anatomical model 348 (e.g., FIG. 19).

In implementations, step 490-11 may include at least partially, or completely, filling the internal cavity 347 with a material (e.g., filler), which may have at least one characteristic that may differs from the first material associated with the first portion 370 and/or the second portion 380. The filler may be removeable from the internal cavity 347 subsequent to formation of the physical anatomical model 348.

At step 490-13, the surgeon or clinical user may position or otherwise prepare the physical anatomical model 148. The physical anatomical model 148 may be secured to at least one fixture 166 to establish an assembly 168 (shown in dashed lines in FIG. 13). The fixture(s) 166 may be arranged relative to a static structure and/or one or more reusable components. The fixture(s) 166 may be representative of surrounding tissue or a portion of a joint. The surgeon may utilize the fixture(s) 166 to simulate rotating or moving a limb in an operating room. Fixture(s) 166 may be representative of skin tissue and may be formed from a relatively flexible material, such as an elastomeric material. The surgeon may form one or more openings in the fixture 166 to simulate performing an incision to expose a joint, bone and/or another portion of the anatomy.

Still referring to FIGS. 2 and 23, one or more modifications to the physical anatomical model(s) may be performed at step 490-11. In implementations, the surgeon or clinical user may use instrumentation to form the internal cavity 374 in the physical anatomical model 348 (FIGS. 19-22), which may occur subsequent to forming the physical anatomical model 348 at step 490-11. The internal cavity 374 may be formed utilizing various techniques, such as a drilling, milling or reaming operation. The surgeon or clinical user may perform various modifications to the physical anatomical model to simulate surgical operations performed on an anatomy to restore functionality to a patient. The simulated surgical operations may include one or more repairs to the anatomy, such as one or more cutting, drilling, reaming, resection and implantation operations. Each modification may result in permanently altering a geometry of the physical anatomical model.

At step 490-15, one or more modifications to the physical anatomical model(s) may be evaluated utilizing any of the techniques disclosed herein. Step 490-15 may include inserting an orthopaedic implant 382 at least partially into the internal cavity 374 (FIGS. 19-22). Step 490-15 may include deploying one or more deployment members 384 of the implant 382 and viewing the deployment through a transparent or translucent material, including through a viewing portal 378 (e.g., window). Step 490-15 may include deploying the deployment member(s) 384 within the deployment region 376.

In the implementation of FIG. 22, step 490-15 may include illuminating the physical anatomical model 348 at step 490-16. Step 490-16 may include illuminating the interior (e.g., interior portion) 3481 of the main body 248M, such as the transparent or translucent material. The interior 3481 of the physical anatomical model 348 may be illuminated with one or more light sources 386. The light source(s) 386 may be at least partially embedded in, or may otherwise be adjacent to, the body 348M physical anatomical model 348. The light source(s) 386 may be arranged to at least partially illuminate the viewing portal 378 and/or the internal cavity 374 including the deployment region 376. Step 490-16 may include illuminating at least the first material associated with the first portion 370 when the implant 382 is in the deployed state.

The novel devices and methods of this disclosure provide versatility in planning, rehearsing and training for surgical procedures utilizing physical anatomical models. The physical anatomical models may be representative of various anatomy, including anatomy associated with various fracture classifications. The surgeon may interact with the disclosed system to gain familiarity with the selected anatomy and various surgical procedures that may be utilized to implement a surgical plan, including the repair of fractures that may be associated with different fracture classifications. The physical anatomical models may be representative of various tissue types and may incorporate transparent or translucent materials. The transparent or translucent portions may assist the surgeon in determining the accuracy and effectiveness of implementing surgical procedures on the physical anatomical model.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. A system for an orthopaedic procedure comprising:
a physical anatomical model including a main body representative of anatomy, wherein the main body includes a bone component, the bone component including an exterior having a surface contour representative of a bone, the bone component comprising:
a first portion establishing the exterior and comprising a first material; and
a second portion adjacent to the first portion, the second portion including a second material that differs from the first material, and the second material is transparent or translucent for viewing an interior of the bone component.

2. The system of claim 1, wherein the second material is transparent.

3. The system of claim 1, wherein the second material is translucent.

4. The system of claim 1, wherein the second material establishes a viewing portal to the interior of the bone component, the interior of the bone component includes an internal cavity at least partially aligned with the viewing portal, the internal cavity dimensioned to at least partially receive an orthopaedic implant.

5. The system of claim 4, wherein the internal cavity extends at least partially through the first portion of the bone component.

6. The system of claim 4, wherein the internal cavity includes a deployment region dimensioned to at least partially receive a portion of the orthopaedic implant movable between an undeployed state and a deployed state, the deployment region at least partially aligned with the viewing portal.

7. The system of claim 6, wherein the deployment region includes a conical void associated with a geometry of the portion of the implant in the deployed state.

8. The system of claim 1, wherein the bone is a fibula, and the second portion is adjacent to an intermediate portion and/or a proximal portion of the bone component.

9. (canceled)

10. (canceled)

11. The system of claim 1, wherein the first material has a transparency that is less than a transparency of the second material, and the first material is substantially opaque.

12. The system of claim 1, wherein the first material has a transparency that is less than a transparency of the second material, and the first portion is distal to the second portion.

13. The system of claim 12, wherein the first portion includes a joint region of the bone component, the joint region representative of an orthopaedic joint.

14. The system of claim 1, further comprising:
a light source at least partially embedded in the main body; and
wherein the light source is operable to illuminate the interior.

15. (canceled)

16. The system as recited in claim 14, wherein the second material establishes a viewing portal to the interior of the bone component, and an opaque material surrounds a perimeter of the viewing portal.

17. The system as recited in claim 1, further comprising:
a soft tissue component on the bone component, wherein the soft tissue component is representative of soft tissue.

18. The system as recited in claim 1, wherein:
the bone component includes a fracture path that establishes one or more localized regions; and
wherein the bone component is severable along the fracture path to establish one or more fragments associated with a respective one of the one or more localized regions.

19. The system of claim 18, wherein the interior of the bone component includes an internal cavity that extends through the fracture path and is at least partially aligned with a viewing portal established by the second material, and the internal cavity is dimensioned to at least partially receive an orthopaedic implant.

20. (canceled)

21. (canceled)

22. (canceled)

23. A method of rehearsing for a surgical procedure comprising:
accessing a virtual anatomical model associated with an anatomy;
generating a configuration associated with a physical anatomical model that is representative of the virtual anatomical model; and
forming the physical anatomical model, the physical anatomical model including a body associated with an anatomical profile of a bone, at least a first portion of the body including a first material that is transparent or translucent for viewing an interior portion of the body.

24. (canceled)

25. The method of claim 23, comprising:
illuminating the interior portion.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A system for an orthopaedic procedure comprising:
a physical anatomical model including a main body representative of anatomy, wherein the main body includes a bone component, the bone component including an exterior having a surface contour representative of a bone, the bone component comprising:
a first portion establishing the exterior and comprising a first material; and
a second portion adjacent to the first portion, the second portion including a second material that differs from the first material, the second material being transparent or translucent for viewing an interior of the bone component, the first material having a transparency that is less than a transparency of the second material, the second material establishing a viewing portal to the interior of the bone component, the interior of the bone component including an internal cavity at least partially aligned with the viewing portal, the internal cavity dimensioned to at least partially receive an orthopaedic implant; and
a light source at least partially embedded in the bone component and operable to illuminate the interior.

38. The system of claim 37, wherein:
the bone component includes a fracture path that establishes one or more localized regions;
the bone component is severable along the fracture path to establish one or more fragments associated with a respective one of the one or more localized regions; and
the internal cavity extends through the fracture path.
